(19) United States
(12) Patent Application Publication
(10) Pub. No.: US 2025/0171468 A1
(43) Pub. Date: May 29, 2025

(54) PHENOXIDO COPPER COMPLEXES AND METHODS FOR USING THEM IN THE TREATMENT OF CANCER

(71) Applicant: UNIVERSITY OF LOUISIANA LAFAYETTE, Lafayette, LA (US)

(72) Inventor: Salah S. MASSOUD, Lafayette, LA (US)

(73) Assignee: UNIVERSITY OF LOUISIANA LAFAYETTE, Lafayette, LA (US)

(21) Appl. No.: 18/958,943

(22) Filed: Nov. 25, 2024

Related U.S. Application Data
(60) Provisional application No. 63/603,412, filed on Nov. 28, 2023.

(51) Int. Cl. C07F 1/08; A61K 33/34

(52) U.S. Cl. CPC C07F 1/08; A61K 33/34

(57) ABSTRACT

Disclosed herein are phenoxido copper compounds, complexes, hydrates, solvates, geometric isomers, or salts thereof, where the compounds have a chemical structure selected from a list: (Cu₂(L¹)₂](ClO₄)₂, (Cu₂(L²)₂](ClO₄)₂, [Cu₂(L³)₂], [Cu₂(L⁴)₂], [Cu₂(L⁴)₂], [Cu₂(L⁵)₂(H₂O)]·2H₂O, [Cu₂(L⁶)₂(H₂O)]·2H₂O, and catena-[Cu(μ-L⁷)]. In one specific embodiment, the method includes administering to a subject a therapeutically effective amount of the phenoxido copper compound.

### CROSS-REFERENCE TO RELATED APPLICATIONS

This application claims benefit of priority under 35 U.S.C. § 119(e) of U.S. Ser. No. 63/603,412, filed Nov. 28, 2023, the entire contents of which is incorporated herein by reference in its entirety.

### BACKGROUND

### Field

Phenoxido copper compounds, complexes, hydrates, solvates, geometric isomers, and salts thereof, wherein the compound has a chemical structure selected from a list: [(Cu₂(L)₂](ClO₄)₂, [(Cu₂(L²)₂](ClO₄)₂, [Cu₂(L³)₂], [Cu₂(L⁴)₂], [Cu₂(L⁴)₂], [Cu₂(L⁵)₂(H₂O)]·2H₂O, [Cu₂(L⁶)₂(H₂O)]·2H₂O, and catena-[Cu(—L⁷)], are described.

### Description of the Related Art

According to WHO cancer is the second leading cause of death for people after heart diseases in 2020 worldwide. It accounts for almost 10 million deaths, of which about 609,360 cancer death were estimated in USA in 2022 by the American Cancer Society [1]. Platinum-containing drugs such as cisplatin, carboplatin and oxaliplatin have been approved worldwide in the treatment of human cancer [2,3]. Although the prevalent success of cisplatin, its second and third generations in the treatment of various types of cancers, many of these compounds suffer serious toxic side effects due to their low selectivity and tumor resistance [2,4]. Serious side effects include nephrotoxicity, neurotoxicity, ototoxicity and toxicities in the kidney and liver, decrease in immunity as well as gastrointestinal disorders. These symptoms limit the use of platinum drugs [4,5]. The clinical relevance of these drugs necessitates the development of new and more efficient anticancer agents that enhance the cytotoxicity and lower the toxic effects. Numerous coordination compounds with different metal ions such as Ru, Os, Ir, Au, Fe, Zn, and Cu coordinated to a wide range of ligand skeletal have emerged in this field as potential antitumor chemotherapeutics [4a,6,7], and these were recently reviewed by Paprocka et al. and Guk et al. [7]. The success of metal-based drugs is associated with the proper choice of the ancillary ligand, which plays a crucial role in modifying the reactivity, stability, and lipophilicity of the complex, and in stabilizing the oxidation states of the central metal ion.

In the last two decades copper complexes were designed and launched as promising frontline agents in the development of effective anticancer chemotherapeutic [8-19]. Copper is an essential element for life as a result it is involved in many biological pathways [20,21]. Thus, the low cost of copper compared to Pt, Au and Ru, and the fact that it is an endogenous metal, which is assumed to be less toxic to normal cells compared to cancer cells, promoted extensive research for designing novel copper-based complexes as an alternative strategic approach for non-platinum drugs. Following this context, large number of copper coordinated to N, O, S-donor ligands were synthesized and their anticancer activities were investigated [8-19,21-27]. Among these complexes [Cu(DDC)₂][22], [Cu(8-HQ)₂][23], [Cu(Pyr)₂][24], [Cu(Plum)₂][25] and [Cu(CQ)₂][26], where DDC=diethyldithiocarbamate, HQ=8-hydroquinoline, Pyr=Pyrithione, Plum=Plumbagin and CQ=clioquinol (5-chloro-7-iodo-8-quinoline), showed IC50 values less than that of cisplatin in all tested cancer cell lines. Similar results (IC50≤6.2 μm) were obtained with the in vitro cytotoxicity of the complexes [Cu(Xi)Cl]ClO₄ and [Cu(X²)Cl]PF₆ (X¹=[bis(2-ethyl-di(3,5-dimethyl-1H-pyrazol-1-yl))-(3,4-dimethoxy-(2-pyr-idylmethyl))]amine, X²=[bis(2-ethyl-di(3,5-dimethyl-1H-pyrazol-1-yl)-(2-quinolymethyl)]amine) against A2780, A2780R, HOS and MCF-7 human cancer cells [16]. In this race the Cu(II) complexes Elesclomol, Casiopeinas IIIia and II-Gly have entered phase I clinical trial for treatment acute myeloid leukemia [27].

Several studies revealed the capability and high affinity of copper ions to interact with enzymes, nuclear proteins, and DNA, where in most cases its interaction with DNA causes site-specific damage [28]. Also, it has been reported that this process delays the cell-cycle progression and increases the cell death in different cell cultures [29]. Moreover, it has been pointed out that copper complexes can interact with DNA without the formation of covalent adducts, but through the noncovalent interactions with the major or minor DNA grooves by intercalation or electrostatic binding. Interestingly, the anoxic character of cancer cells cannot stabilize Cu(II) oxidation state, and as a result it promotes the reduction of Cu(II) to Cu(I), a process which does not occur in normal cells, and thus producing Cu(I) as the reactive species to target the tumor cells [30]. This redox process leads to the release of Cu(I), which in turn catalyzes the formation of reactive oxygen species (ROS) to induce a pro-apoptotic oxidative stress for the cancer cells [30].

Consequently, there is a need for new copper compounds that can provide effective anticancer chemotherapeutics.

### SUMMARY

Provided herein are phenoxido copper compounds, complexes, hydrates, solvates, geometric isomers, and salts thereof that can be used to treat one or more types of cancers. In one specific embodiment, the phenoxido copper complex has a chemical structure selected from a list: [(Cu₂(L¹)₂](ClO₄)₂; [(Cu₂(L²)₂](ClO₄)₂; [Cu₂(L³)₂]; [Cu₂(L⁴)₂]; [Cu₂(L⁴)₂][Cu₂(L⁵)₂(H₂O)]2H₂O; [Cu₂(L⁶)₂(H₂O)]2H₂O; catena-[Cu(—L⁷)], where L¹⁻⁷ is a ligand with a chemical structure:

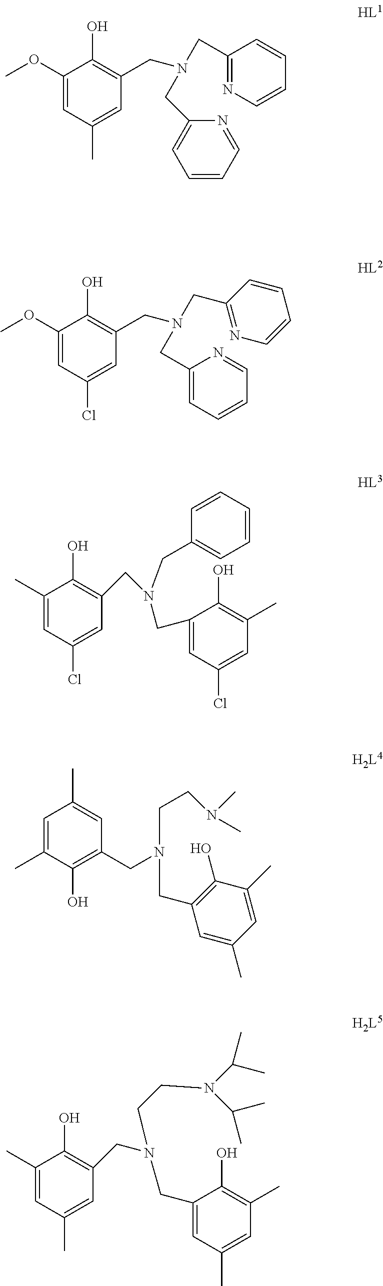

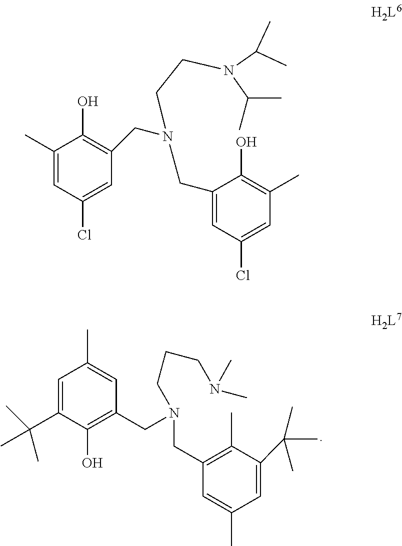

In another specific embodiment, a method for treating cancer, where the method includes administering to a subject a therapeutically effective amount of the phenoxido copper compound.

### BRIEF DESCRIPTION OF THE DRAWINGS

For the purpose of promoting an understanding of the principles of the present disclosure, reference is now made to the embodiments illustrated in the drawings, which are described below. The embodiments disclosed herein are not intended to be exhaustive or limit the present disclosure to the precise form disclosed in the following detailed description. Rather, the embodiments are chosen and described so that others skilled in the art can utilize their teachings. Therefore, no limitation of the scope of the present disclosure is thereby intended.

FIG. 1 shows the chemical formula for the ligands L-⁷ used in the phenoxido copper complexes.

FIG. 2 is a molecular plot of [Cu₂(L¹)₂]²⁺ complex cation of 1.

FIG. 3 is a molecular plot of [Cu₂(L²)₂]²⁺ complex cation of 2.

FIG. 4 is a molecular plot of [Cu₂(L³)₂] complex of 3.

FIG. 5 is a molecular plot of [Cu₂(L⁴)₂] complex of 4.

FIG. 6 is a molecular plot of [Cu₂(L⁵)₂(H₂O)]2H₂O complex of 5.

FIG. 7 is a molecular plot of [Cu₂(L⁶)₂(H₂O)]2H₂O complex of 6.

FIG. 8 is a molecular plot of catena-[Cu₂(p-L⁷)] complex of 7.

FIG. 9 shows Table 10f the in vitro cytotoxicity data obtained for the phenoxido copper complexes 1-7 and a reference drug cisplatin against human cancer cell lines (A2780, A2780R, MCF-7, HOC-1, CaCo2, PC-3, 22Rbl, and HeLa) and normal healthy fibroblasts MRC-5 expressed as IC₅₀ values in μM±standard deviation calculated from dose-response curves in three independent determinations after 24 h of incubation.

FIG. 10 is a graph of the levels of intracellular copper of compound 6 determined by ICP-MS method in A2780 after incubation at 2, 6, 12, 24, 48 and 72 h.

FIG. 11 is a bar graph of the cell-cycle modifications caused by the complexes 4, 6, and 7 applied to A2780 cells at 3, 3 and 15 M concentrations, respectively, for 24 h of incubation. The results represent the calculated mean values of relative numbers of cell populations (GO/G1, S, and G2/M)±standard deviation from three parallel determinations.

FIG. 12 is a bar graph showing the induction of cell death in A2780 cells promoted by the incubation with compounds 4 (applied at 3 μM), 6 (at 3 μM) and 7 (at 15 μM) and compared to the reference drug cisplatin (applied at 19 M concentration).

FIG. 13 is a bar graph showing the activation of effector caspases 3/7 in A2780 cells after 24 h incubation with copper(II) compounds 4, 6, and 7, and reference drug cisplatin applied at the IC50 concentrations obtained from cytotoxicity screening. The results represent means±SD from three independent experiments.

FIG. 14 is a bar graph showing the results of autophagy detection in A2780 cells incubated for 24 h with complexes 4 (3 μM), 6 (3 μM), and 7 (15 M) and a reference drug cisplatin (19M). The CQ/Rapamycin treatment was used as a positive standard showing the maximum autophagic flux as compared to untreated control cells.

FIG. 15 is a bar graph showing the detection of reactive oxygen species production in A2780 cells after the 24 h incubation with the tested compounds (applied at IC50 concentrations as obtained in cytotoxicity testing). The groups designated as Pyocyanin−(negative) were not pretreated with pyocyanin, and the groups marked as Pyocyanin+(positive) were pretreated with pyocyanin, a known oxidative stress inducer over 24 h. The results represent fluorescence signal of the detection probe for ROS obtained from three parallel experiments.

FIG. 16 is a bar graph showing the detection of superoxide radical production in A2780 cells after the 24 h incubation with the tested compounds (applied at IC50 concentrations as obtained in cytotoxicity testing). The groups designated as Pyocyanin−(negative) were not pretreated with pyocyanin, and the groups marked as Pyocyanin+(positive) were pretreated with pyocyanin, a known oxidative stress inducer over 24 h. The results represent fluorescence signal of the detection probe for ROS obtained from three parallel experiments.

FIG. 17 is a bar graph showing the results of mitochondrial membrane destruction determined by means of the mitochondrial membrane depolarization kit representing the number of cells with damaged mitochondrial membrane in A2780 cells after 24 h incubation with compounds 4, 6, and 7 and a reference drug cisplatin (applied at the IC50 concentrations obtained in cytotoxicity screening). The results represent mean values±SD from three parallel experiments.

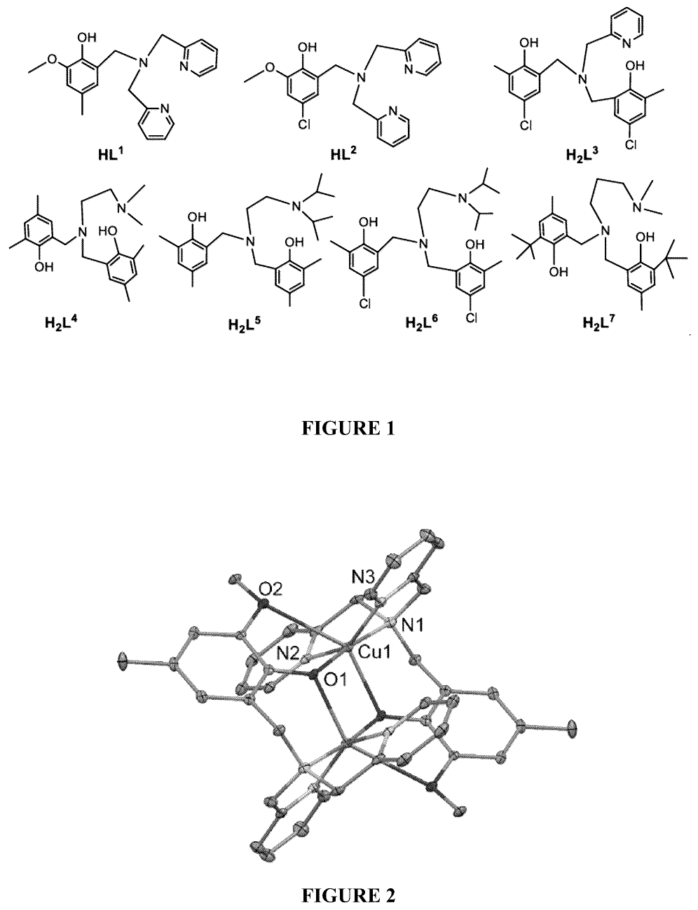

### DETAILED DESCRIPTION

In one or more embodiments, the phenoxido copper compounds and/or complexes can include, but are not limited to, compounds with a chemical structure selected from a list: (Cu₂(L¹)₂](ClO₄)₂; (Cu₂(L²)₂](ClO₄)₂; [Cu₂(L³)₂]; [Cu₂(L⁴)₂]; [Cu₂(L⁴)₂]; [Cu₂(L⁵)₂(H₂O)]2H₂O; [Cu₂(L⁶)₂(H₂O)]2H₂O; catena-[Cu(—L⁷)], where L-⁷ is a ligand with a chemical structure:

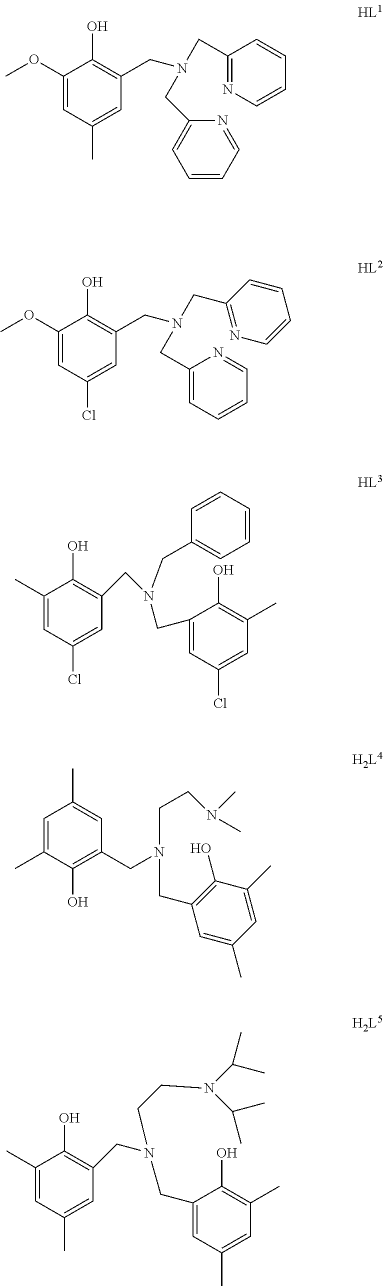

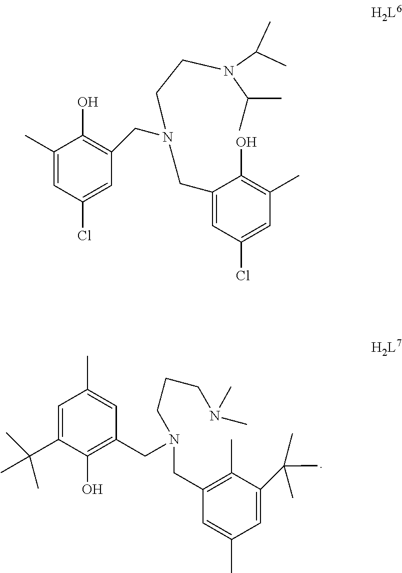

In some embodiments, the central atom can include but, is not limited to: Cu(I), Cu(II), and mixtures thereof. In some embodiments, the phenoxido copper complexes can include, but are not limited to: two cationic compounds and/or complexes [Cu₂(L¹⁻²)₂](ClO₄)₂ (1, 2), and four neutral doubly bridged-phenoxido-copper(II) compounds, complexes [Cu₂(L³⁻⁴)₂](3, 4), [Cu₂(L⁵⁻⁶)₂(H₂O)]·2H₂O (5, 6), and 1D polymeric catena-[Cu(L⁷)](7), where HL¹⁻² and H₂L³⁻⁷ represent tripodal tetradentate pyridyl or aliphatic-amino groups based 2,4-disubstituted phenolates. In some embodiments, the molecular structures of the phenoxido copper complexes can exhibit diverse geometrical environments around the central Cu(II) atoms. The in vitro antiproliferative activity of the isolated complexes and selected parent free ligands were screened against some human cancer cell lines (A2780, A2780R, PC-3, 22Rvl, MCF-7). The most promising cytotoxicity against cancer cells were obtained for 1-6, while complex 6 was found as the best performing as compared to the reference drug cisplatin. The cytotoxicity study of complex 6 was therefore extended to wider variety of cancer cell lines (HOS, A549, PANC-1, CaCo₂, HeLa) and results revealed its significant cytotoxicity on all investigated human cancer cells. The cell uptake study showed that cytotoxicity of 6 (3 M concentration and 24 h of incubation) against A2780 cells was almost independent from the intracellular levels of copper. The effect of compounds 4, 6 and 7 on cell cycle of A2780 cells indicates that the mechanism of action in these complexes is not only different from that of cisplatin but also different among them. Complex 7 was able to induce apoptosis in A2780 cells, while complexes 4 and 6 did not and on the other hand, they showed considerable effect on autophagy induction and there are some clues that these complexes were able to induce cuproptosis in A2780 cells.

In one or more embodiments, a method for treating a cancer in a subject can include, but is not limited to: administering to a subject a therapeutically effective amount of a phenoxido copper complex or a pharmaceutically acceptable salt of the complex with a chemical structure selected from a list: [(Cu₂(L¹)₂](ClO₄)₂; [(Cu₂(L²)₂](ClO₄)₂; [Cu₂(L³)₂]; [Cu₂(L⁴)₂]; [Cu₂(L⁴)₂]; [Cu₂(L⁵)₂(H₂O)]2H₂O; [Cu₂(L⁶)₂(H₂O)]·2H₂O; catena-[Cu(—L⁷)].

In one or more embodiments, a method for making the one or more phenoxido copper complexes can include but is not limited to: contacting connecting a 2-,4-disubstituted phenol with an amine to make a ligand, and contacting connecting the ligand with one or more copper salts ion to make a phenoxido copper complex. The one or more copper salts can include but, are not limited to: Cu(NO₃)₂ and Cu(ClO₄)₂.

The phenoxido copper complexes can be provided in many forms. For example, the phenoxido copper complexes can include, but are not limited to, salts, hydrates, solvates, isomers, crystalline and non-crystalline forms, isomorphs, polymorphs, and metabolites thereof. The phenoxido copper complexes may exist in unsolvated and solvated forms. For example, when the solvent, such as water, is tightly bound, the complex will have a well-defined stoichiometry independent of humidity. When, however, the water is weakly bound, as in channel solvates and hygroscopic compounds, the water/solvent content can depend on humidity and drying conditions; hence, non-stoichiometry will be the norm. Moreover, the phenoxido copper complexes may exist in unsolvated as well as solvated forms with pharmaceutically acceptable solvents, such as water and methanol. In general, the solvated forms are considered equivalent to the unsolvated forms. The phenoxido copper complexes may also exist in one or more crystalline states, i.e., polymorphs, or they may exist as amorphous solids.

In some embodiments, the ligands for the phenoxido copper compound or complex can include, but are not limited to, ligands with a chemical structure:

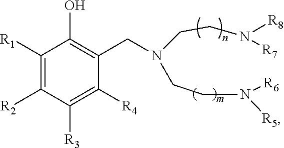

where R₁, R₂, R₃, R₄, R₅, R₆, R₇, and R₈ can be independently selected from the group consisting of: H; (C₂₋₈)alkyl, (C₂₋₈)alkenyl, (C₂₋₈)alkenyl, F; Cl; Br; I; N; 0; OH; ketone (═O); ether [—OR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; acyl halide (—COX); carbonyl [—COR, where R can include methyl, ethyl, n-propyl, isopropyl, n-butyl, sec-butyl, isobutyl, tert-butyl; (C2-4)alkenyl, such as ethenyl, propenyl, butenyl, where the double bond can be located at any position in the alkenyl carbon chain, and including any alkenyl conformational isomers]; aldehyde (—CHO); carbonate ester [—OCOOR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; carboxyl (—COOH); amide [—CONR′R″, where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; amines [—NR′R″, where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; cyanate (—OCN); isocynate (—NCO); nitrate (—ONO₂); nitrile (—CN); isonitrile (—NC); nitroso (—NO); oxime (—CH═NOH); borono B(OH)₂; boronare [—B(OR′)(R″), where R′ and R″ can be independently selected from hydrogen, methyl, ethyl, n-propyl, isopropyl, n-butyl, sec-butyl, isobutyl, tert-butyl; (C₂4)alkenyl, such as ethenyl, propenyl, butenyl, where the double bond can be located at any position in the alkenyl carbon chain, and including any alkenyl conformational isomers]; borinate [—B(R′)(OR″), where R′ and R″ can be independently selected from hydrogen, methyl, ethyl, n-propyl, isopropyl, n-butyl, sec-butyl, isobutyl, tert-butyl; (C₂4)alkenyl, such as ethenyl, propenyl, butenyl, where the double bond can be located at any position in the alkenyl carbon chain, and including any alkenyl conformational isomers]; phosophino [—PR₂, where R can include hydrogen, methyl, ethyl, n-propyl, isopropyl, n-butyl, sec-butyl, isobutyl, tert-butyl; (C₂4)alkenyl, such as ethenyl, propenyl, butenyl, where the double bond can be located at any position in the alkenyl carbon chain, and including any alkenyl conformational isomers]; phosphono [—P(═O)(OH)(R), where R can include hydrogen, methyl, ethyl, n-propyl, isopropyl, n-butyl, sec-butyl, isobutyl, tert-butyl; (C24)alkenyl, such as ethenyl, propenyl, butenyl, where the double bond can be located at any position in the alkenyl carbon chain, and including any alkenyl conformational isomers]; phosphate (—OP(═O)(OH)₂; thiol (—SH); sulfide [—SR, where R can include methyl, ethyl, n-propyl, isopropyl, n-butyl, sec-butyl, isobutyl, tert-butyl; (C₂₄)alkenyl, such as ethenyl, propenyl, butenyl, where the double bond can be located at any position in the alkenyl carbon chain, and including any alkenyl conformational isomers]; disulfide [—SSR, where R can include methyl, ethyl, n-propyl, isopropyl, n-butyl, sec-butyl, isobutyl, tert-butyl; (C₂-₄)alkenyl, such as ethenyl, propenyl, butenyl, where the double bond can be located at any position in the alkenyl carbon chain, and including any alkenyl conformational isomers], sulfinyl [—S(═O)R, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; sulfino (—SO₂H); sulfo (—SO₃H); thiocyanate; isothiocyanate; carbonothioyl [—C(═S)R where R can include methyl, ethyl, n-propyl, isopropyl, n-butyl, sec-butyl, isobutyl, tert-butyl; (C₂-₄)alkenyl, such as ethenyl, propenyl, butenyl, where the double bond can be located at any position in the alkenyl carbon chain, and including any alkenyl conformational isomers]; (C₁₋₄)alkyl, such as methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers; where n can include an integer from 1 to 10; and where m can include an integer from 1 to 10.

Many of the phenoxido copper complexes may exist as geometric isomers or possess one or more asymmetric centers, thus existing as two or more stereoisomeric forms. The phenoxido copper complexes can include all the individual stereoisomers and geometric isomers of the compounds and mixtures thereof. Individual enantiomers can be obtained by chiral separation or using the relevant enantiomer in the synthesis.

The phenoxido copper complexes can include asymmetric carbons. When present in racemic compounds, solid and dotted wedges are used to define relative stereochemistry, rather than absolute stereochemistry. Racemic compounds possessing such indicated relative stereochemistry are marked with (+/−). Unless stated otherwise, it is intended that the phenoxido copper complexes can exist as stereoisomers, which include cis and trans isomers, optical isomers such as R and S enantiomers, diastereomers, geometric isomers, rotational isomers, conformational isomers, atropisomers, and mixtures thereof (such as racemates and diastereomeric pairs). The phenoxido copper complexes may exhibit more than one type of isomers in solution. Also included are acid addition or base addition salts wherein the counterion is optically active, for example, D-lactate or L-lysine, or racemic, for example, DL-tartrate or DL-arginine. When any racemate crystallizes, crystals of two different types are possible. The first type is the racemic compound (true racemate) where one homogeneous form of crystal is produced containing both enantiomers in equimolar amounts. The second type is the racemic mixture or conglomerate wherein two forms of crystal are produced in equimolar amounts each comprising a single enantiomer.

The phenoxido copper complexes can also include prodrugs. Thus, certain phenoxido copper complexes that may have little or no pharmacological activity themselves can, when administered into or onto the body, be converted into a compound having the desired activity, for example, by hydrolytic cleavage. Further information on the use of prodrugs may be found in “Pro-drugs as Novel Delivery Systems,” Vol. 14, ACS Symposium Series (T. Higuchi and W. Stella) and “Bioreversible Carriers in Drug Design,” Pergamon Press, 1987 (ed. E. B. Roche, American Pharmaceutical Association). Prodrugs can, for example, be produced by replacing appropriate functionalities present in the phenoxido copper complexes with certain moieties known to those skilled in the art as “pro-moieties” as described, for example, in “Design of Prodrugs” by H. Bundgaard (Elsevier, 1985).

The phenoxido copper complexes can include all pharmaceutically acceptable isotopically labeled compounds, which are identical to those recited herein, wherein one or more atoms are replaced by an atom having the same atomic number, but an atomic mass or mass number different from the atomic mass or mass number which predominates in nature. Examples of isotopes suitable for inclusion in the phenoxido copper complexes can include, but are not limited to, isotopes of hydrogen, such as ²H, ³H; carbon, such as ¹¹C, ¹³C, and ¹⁴C; chlorine, such as ³⁶Cl; fluorine, such as ¹⁸F; iodine, such as ¹²³I and ¹²⁵I; nitrogen, such as ¹³N and ¹⁵N; oxygen, such as ¹⁵O, ¹⁷O, and ¹⁸O; phosphorus, such as ³²P; and sulfur, such as ³⁵S. Certain isotopically-labeled phenoxido copper complexes, for example, those incorporating a radioactive isotope, are useful in drug and/or substrate tissue distribution studies (e.g., assays). The radioactive isotopes tritium, i.e., ³H, and carbon-14, i.e., ¹⁴C, are particularly useful for this purpose in view of their ease of incorporation and ready means of detection. Substitution with heavier isotopes such as deuterium, i.e., ²H, may afford certain therapeutic advantages resulting from greater metabolic stability, for example, increased in vivo half-life or reduced dosage requirements and, hence, may be preferred in some circumstances. Substitution with positron-emitting isotopes, such as ¹¹C, ¹⁵F, ¹⁸F, ¹⁵O and ¹³N, can be useful in positron emission tomography studies for examining substrate receptor occupancy. Isotopically labeled phenoxido copper complexes can generally be prepared by conventional techniques or by processes analogous to those described in Scheme 1 or the Examples (see below) using an appropriate isotopically labeled reagent in place of the non-labeled reagent previously employed. Pharmaceutically acceptable solvates can include those wherein the solvent of crystallization may be isotopically substituted, e.g., D₂O, acetone-d₆, or DMSO-d₆. Phenoxido copper complexes can include, but are not limited to, isotopically labeled versions of these compounds, such as, but not limited to, the deuterated and tritiated isotopes and all other isotopes discussed above.

The phenoxido copper complexes and/or compounds can be provided in the form of salts derived from inorganic and/or organic compounds acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of the phenoxido copper complexes may be used as an aid in the isolation, purification, and/or resolution of the compound. When a salt is intended to be administered to a subject the salt can be provided in a pharmaceutically acceptable form. A pharmaceutical salt compound can be prepared by combining a phenoxido organic ligand with a copper salt. complex with an acid whose anion, or a base whose cation, is generally considered suitable for mammalian consumption

Suitable pharmaceutically salts of the phenoxido copper complexes can include those derived from inorganic acids. The inorganic acids can include, but are not limited to, hydrochloric, hydrobromic, hydrofluoric, boric, fluoroboric, phosphoric, meta-phosphoric, nitric, carbonic, sulfonic, and sulfuric acids.

Suitable pharmaceutical salts of the phenoxido copper complexes can include those derived from organic acids. The organic acids can include, but are not limited to, aliphatic, cycloaliphatic, aromatic, aliphatic, heterocyclic, carboxylic, and sulfonic classes of organic acids. Specific examples of suitable organic acids can include, but are not limited to: acetate, trifluoroacetate, formate, propionate, succinate, glycolate, gluconate, digluconate, lactate, malate, tartrate, citrate, ascorbate, glucuronate, maleate, fumarate, pyruvate, aspartate, glutamate, benzoate, anthranilate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sulfonates, cyclohexylamino-ethansulfonate, algenic acid, p-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalene-sulfonate, oxalate, palmoate, pectinate, 3-phenylpropionate, picrate, pivalate, thiocyanate, and undecanoate.

Organic salts of the phenoxido copper complexes may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl(C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), and arylalkyl halides (e.g., benzyl and phenethyl bromides).

Furthermore, when the phenoxido copper complexes carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

The phenoxido copper compounds and/or complexes can be made by using many synthetic strategies and known chemical transformations. The reaction Scheme 1, together with synthetic methods known in the art of organic chemistry, or modifications and derivatizations that are familiar to those of ordinary skill in the art, illustrate a method for preparing the phenoxido copper complexes. The starting materials used can be commercially available or may be prepared by routine methods known in the art. For example, the starting materials can be prepared by the methods discussed and described in the COMPENDIUM OF ORGANIC SYNTHETIC METHODS, Vol. I-XII (published by Wiley-Interscience).

One skilled in the art will recognize that in some cases, the phenoxido copper complexes can be made by various synthetic strategies. and chemical transformations will be generated as a mixture of diastereomers and/or enantiomers; these may be separated at various stages of the synthetic scheme using conventional techniques or a combination of such techniques, such as, but not limited to, crystallization, normal-phase chromatography, reversed phase chromatography and chiral chromatography, to afford the pure desired product single enantiomers of the phenoxido copper complexes.

The phenoxido copper complexes and/or compounds can be incorporated into pharmaceutical compositions. For example, a pharmaceutical composition can include a mixture of one or more phenoxido copper complexes and one or more pharmaceutically acceptable carriers. In another example, a pharmaceutical composition can include a mixture of one or more phenoxido copper complexes, one or more pharmaceutically acceptable carriers, and one or more adjuvants. Other pharmacologically active substances can also be present. The phenoxido copper complexes can be administered as a compound per se or the phenoxido copper compunds can be administered as pharmaceutically acceptable salts. Pharmaceutically acceptable salts of phenoxido copper complexes can have greater aqueous solubility relative to the parent compound.

Compositions that include phenoxido copper complexes can be formulated according to known methods for preparing pharmaceutically useful compositions. Formulations are described in detail in a number of sources which are well known and readily available to those skilled in the art. For example, Remington's Pharmaceutical Science by E. W. Martin describes how to make pharmaceutical formulations. In general, the compositions that include phenoxido copper complexes can be formulated such that an effective amount of at least one phenoxido copper complex is combined with a suitable pharmaceutically acceptable carrier or diluent in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art.

The pharmaceutically acceptable carriers or diluents for use with phenoxido copper complexes can include, but are not limited to: water, saline, oils including mineral oil, ethanol, dimethyl sulfoxide, gelatin, cyclodextrans, magnesium stearate, dextrose, cellulose, sugars, calcium carbonate, glycerol, alumina, starch, and equivalent carriers and diluents, or mixtures of any of these.

The adjuvants can include, but are not limited to, wetting agents, stabilizing agents, binding agents, dispersing agents, emulsifying agents, suspending agents, bioadhesive agents, polymers, and flavoring agents. The preservative can include, but is not limited to, benzalkonium chloride. The polymer can include, but is not limited to, polyacrylic acid, polyvinyl alcohol, hyaluronic acid, hydroxypropylmethyl cellulose, hydroxyethyl cellulose, methyl cellulose, and heteropolysaccharide polymers, such as gelan gum.

To provide for the administration of such dosages for the desired therapeutic treatment, pharmaceutical compositions of the phenoxido copper complexes can be from a low of about 0.05% and to a high of about 99.9% by weight of the total of the one or more of the phenoxido copper complexes based on the weight of the total composition, including a pharmaceutically acceptable carrier and any adjuvant. For example, the pharmaceutical compositions can have phenoxido copper complexes from about 0.05 wt % to about 99.9 wt %, 0.05 wt % to about 10 wt %, 0.2 wt % to about 5 wt %, 1 wt % to about 20 wt %, 5 wt % to about 45 wt %, 8 wt % to about 65 wt %, or 15 wt % to about 85 wt %, based on the weight of the total composition, including any pharmaceutically acceptable carrier and/or adjuvant.

The pharmaceutically acceptable carriers can be a solid, a liquid, or both, and may be formulated with the compound as a unit-dose composition, for example, a tablet, which can contain from a low of about 0.05 wt % to a high of about 99.9 wt % of the active compounds. The pharmaceutical compositions of phenoxido copper complexes can be coupled with suitable polymers as targetable pharmaceutically acceptable carriers.

Other pharmaceutically acceptable carriers and modes of administration known in the pharmaceutical art may also be used. Pharmaceutical compositions of phenoxido copper complexes may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

The phenoxido copper complexes can be used alone or in combination with other therapeutic and/or active compounds in the treatment of various conditions or disease states. The phenoxido copper complexes and other compounds may be administered simultaneously (either in the same dosage form or in separate dosage forms) or sequentially. The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration. The phrases “concurrent administration,” “co-administration,” “simultaneous administration,” and “administered simultaneously” mean that the compounds are administered in combination.

The phenoxido copper complexes may be administered by any suitable route, preferably in a form of a pharmaceutical composition adapted to such a route, and in a dose effective for the treatment intended. The active compounds and compositions, for example, may be administered orally, rectally, parenterally, or topically (e.g., intranasal or ophthalmic).

The phenoxido copper complexes and compositions thereof can be administered to a subject orally, topically, and/or by injection. Oral administration may involve swallowing, so that the compound enters the gastrointestinal tract, or buccal or sublingual administration may be employed, by which the compound enters the blood stream directly from the mouth.

The phenoxido copper complexes may be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

The phenoxido copper complexes can be formulated such that administration topically to the skin or mucosa (i.e., dermally or transdermally) leads to systemic absorption of the compounds. The phenoxido copper complexes can also be formulated such that administration intranasally or by inhalation leads to systemic absorption of the compound.

The dosage regimen for the phenoxido copper complexes can be based on a variety of factors, including the type, age, weight, sex and medical condition of the patient; the severity of the condition; the route of administration; and the activity of the particular compound employed. Thus, the dosage regimen may vary widely. Dosage levels of the order from about 0.01 mg to about 100 mg per kilogram of body weight per day are useful in the treatment of the above-indicated conditions. The total daily dose of a phenoxido copper complexes (administered in single or divided doses) is typically from about 0.01 mg/kg to about 100 mg/kg. In another embodiment, the total daily dose of the phenoxido copper complex is from about 0.1 mg/kg to about 50 mg/kg, and in another embodiment, from about 0.5 mg/kg to about 30 mg/kg (i.e., mg phenoxido copper complex per kg body weight). In some embodiments, dosing is from about 0.01 mg/kg/day to about 10 mg/kg/day. In another embodiment, dosing is from about 0.1 mg/kg/day to about 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day may be used to increase the total daily dose, if desired. Intravenously, doses may range from about 0.1 to about 10 mg/kg/minute during a constant rate infusion.

Oral administration of a solid dose form can be presented in discrete units, such as hard or soft capsules, pills, cachets, lozenges, or tablets, each containing a predetermined amount of at least one of the phenoxido copper complexes. In another embodiment, the oral administration may be in a powder or granule form. In another embodiment, the oral dose form is sub-lingual, such as, for example, a lozenge. In such solid dosage forms, the phenoxido copper complexes are ordinarily combined with one or more adjuvants. Such capsules or tablets may contain a controlled-release formulation. In the case of capsules, tablets, and pills, the dosage forms also may comprise buffering agents or may be prepared with enteric coatings.

For oral administration, the phenoxido copper complexes can be provided in the form of tablets containing from about 0.01 mg to about 0.05 mg, about 0.1 mg to about 0.5 mg, about 1.0 mg to about 2.5 mg, about 5.0 mg to about 10.0 mg, about 15.0 mg to about 25.0 mg, about 50.0 mg to about 75.0 mg, about 100 mg to about 125 mg, 150 mg to about 175 mg, about 200 mg to about 250 mg, and 0.01 mg to about 500 mg of the active ingredient for the symptomatic adjustment of the dosage to the patient. In another example, the phenoxido copper complexes can be provided in the form of tablets containing from about 0.05 wt % to about 99.9 wt %, 0.05 wt % to about 10 wt %, 0.2 wt % to about 5 wt %, 1 wt % to about 20 wt %, 5 wt % to about 45 wt %, 8 wt % to about 65 wt %, or 15 wt % to about 85 wt % of phenoxido copper complexes, based on the weight of the total composition, including any pharmaceutically acceptable carrier and/or adjuvant.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). For example, the phenoxido copper complexes can be provided in a liquid form containing from about 0.05 wt % to about 99.9 wt %, 0.05 wt % to about 10 wt %, 0.2 wt % to about 5 wt %, 1 wt % to about 20 wt %, 5 wt % to about 45 wt %, 8 wt % to about 65 wt %, or 15 wt % to about 85 wt % of phenoxido copper complexes, based on the weight of the total composition, including any pharmaceutically acceptable carrier and/or adjuvant.

In another embodiment, the pharmaceutical compositions of the phenoxido copper complexes can include a parenteral dose form. Parenteral administration can include, but is not limited to, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using suitable dispersing, wetting, and/or suspending agents, and include depot formulations.

The phenoxido copper complexes can be administered in a topical dose form. Topical administration can include, but is not limited to, transdermal administration, such as via transdermal patches or iontophoresis devices, intraocular administration, or intranasal or inhalation administration. Compositions for topical administration can include, but is not limited to, topical gels, sprays, ointments, and creams. A topical formulation can include a compound that enhances absorption or penetration of the active ingredient through the skin or other affected areas. When the phenoxido copper complexes are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes can also be used. Typical carriers include alcohol, water, mineral oil, liquid petrolatum, white petrolatum, glycerin, polyethylene glycol and propylene glycol. Penetration enhancers may be incorporated-see, for example, Finnin and Morgan, J. Pharm. Sci., 88 (10), 955-958 (1999).

For intranasal administration or administration by inhalation, the active phenoxido copper complexes are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone; as a mixture, for example, in a dry blend with lactose; or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may include a bio adhesive agent, for example, chitosan or cyclodextrin.

The phenoxido copper complexes can be used in kits that are suitable for use in performing the methods of treatment described above. In some embodiments, the kit contains a first dosage form comprising one or more of the compounds of the present invention and a container for the dosage, in quantities sufficient to carry out the methods of the present invention.

The phenoxido copper complexes can also be administered utilizing liposome technology, slow-release capsules, implantable pumps, and biodegradable containers. These delivery methods can, advantageously, provide a uniform dosage over an extended period of time.

Various techniques may be used to increase bioavailability of the phenoxido copper complexes. For example, prodrugs of phenoxido copper complexes can be prepared. Prodrugs employ various physical and chemical modifications to improve features of the active drug, and in some embodiments may be viewed as pharmacologically inactive prodrug functional groups that undergo a chemical transformation or enzymatic cleavage to liberate the active parent drug and produce the desired effect in the body. Utilizing a prodrug approach can yield benefits such as enhanced solubility, improved selective targeting of drugs to anatomical sites, protection from rapid metabolism and elimination, reduction toxic effects of an active drug on other parts of the body, and enhanced patient compliance.

Non-limiting examples of techniques useful for enhancing the bioavailability of phenoxido copper complexes can include the use of co-solvents, hydrotropy, micronization, change in dielectric constant of solvent, amorphous forms, chemical modification of the drug, use of surfactants, inclusion complex, alteration of pH of solvent, use of hydrates or solvates, use of soluble prodrugs, application of ultrasonic waves, functional polymer technology, controlled precipitation technology, evaporative precipitation in aqueous solution, use of precipitation inhibitors, solvent deposition, precipitation, selective adsorption on insoluble carriers, size reduction technologies, lipid based delivery systems, micellar technologies, porous micro particle technology, solid dispersion technique, and various types of solid dispersion systems.

Other methods for enhancement of bioavailability, such as by the enhancement of solubility, are described in Reddy M. S. et al., “Solubility enhancement of fenofibrate, a BCS class II drug, by self-emulsifying drug delivery systems,” International Research Journal of Pharmacy, 2011, 2(11): 173-177; Khamkar G S, “Self-micro emulsifying drug delivery system (SMEED) o/w microemulsion for BCS Class II drugs: an approach to enhance oral bioavailability,” International Journal of Pharmacy and Pharmaceutical Sciences, 2011, 3(3):1-3; Elgart A et al., “Improved oral bioavailability of BCS class 2 compounds by self-nano-emulsifying drug delivery systems (SNEDDS): the underlying mechanisms for amiodarone and talinolol”, Pharm Res., 2013 December; 30(12):3029-44; Singh N. et al., “Techniques for bioavailability enhancement of BCS class II drugs: a review,” International Journal of Pharmaceutical and Chemical Science, 2013, 2(2):1092-1101; Elkihel L. et al., “Synthesis and orally macrofilaricidal evaluation of niclosamide lymphotropic prodrugs,” Arzneimittelforschung, 1994, 44(11): 1259-64; and Kansara H. et al., “Techniques used to enhance bioavailability of BCS class II drugs: a review,” Int. J. Drug Dev. & Res., 2015, 7(1):82-93.

The method of using the phenoxido copper complexes can include, but is not limited to, packaged dosage formulations and kits. A packaged dosage formulation can include one or more containers of one or more phenoxido copper complexes formulated in a pharmaceutically acceptable dosage. The package can contain discrete quantities of the dosage formulation, such as tablet, capsules, lozenge, and powders. The quantity of compound in a dosage formulation and that can be administered to a patient can vary from about 1 mg to about 5000 mg, about 1 mg to about 2000 mg, about 1 mg to about 500 mg, about 5 mg to about 250 mg, or about 10 mg to about 100 mg.

The kits can be packaged into suitable packaging material, optionally in combination with instructions for using the kit components, e.g., instructions for using phenoxido copper complexes. In some embodiments, a kit includes an amount of at least of the phenoxido copper complexes, and instructions for administering at least of the phenoxido copper complexes to a subject in need of treatment on a label or packaging insert. In further embodiments, a kit includes an article of manufacture, for delivering at least one or the phenoxido copper complexes into a subject locally, regionally or systemically.

As used herein, the term ‘packaging material’ refers to a physical structure housing the components of the kit. The packaging material can maintain the components in a sterile state, and can be made of material commonly used for such purposes (e.g., paper, corrugated fiber, glass, plastic, foil, ampules, etc.). The label or packaging insert can include appropriate printed and/or digital instructions, for example, for practicing a method of the invention. Thus, in additional embodiments, a kit includes a label or packaging insert including instructions for practicing a method of the invention in solution, in vitro, in vivo, or ex vivo.

Instructions can therefore include instructions for practicing any of the methods of using phenoxido copper complexes described herein. For example, pharmaceutical compositions can be included in a container, pack, or dispenser together with instructions for administration to a subject to treat a disease. Instructions may additionally include indications of a satisfactory clinical endpoint or any adverse symptoms that may occur, storage information, expiration date, or any information required by regulatory agencies such as the Food and Drug Administration or European Medicines Agency for use in a human subject.

The instructions may be digital or on “printed matter,” e.g., on paper or cardboard within the kit, on a label affixed to the kit or packaging material or attached to a vial or tube containing a component of the kit. Instructions may comprise voice or video tape and additionally be included on a computer readable medium, such as a disk (diskette or hard disk), optical CD such as CD- or DVD-ROM/RAM, magnetic tape, electrical storage media such as RAM and ROM and hybrids of these such as magnetic/optical storage media.

Each component of the kit can be enclosed within an individual container or in a mixture, and all of the various containers can be within single or multiple packages. Kits can include packaging material that is compartmentalized to receive one or more containers such as vials, tubes, and the like, each of the container(s) including one of the separate elements to be used in a method described herein. Packaging materials for use in packaging pharmaceutical products include, by way of example only U.S. Pat. Nos. 5,323,907, 5,052,558 and 5,033,252. Examples of pharmaceutical packaging materials can include, but are not limited to, blister packs, bottles, tubes, pumps, bags, vials, light-tight sealed containers, syringes, bottles, and any packaging material suitable for a selected formulation and intended mode of administration and treatment.

A kit may include one or more additional containers, each with one or more of various materials desirable from a commercial and user standpoint for use of the compounds for treating or preventing diseases. Non-limiting examples of such materials include, but not limited to, buffers, diluents, carrier, package, container, vial and/or tube labels listing contents and/or instructions for use, and package inserts with instructions for use.

A label can be on or associated with a container containing a phenoxido copper complexes. A label can be on a container when letters, numbers or other characters forming the label are attached, molded, or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In some embodiments of the kit, the phenoxido copper complexes can be presented in a pack or dispenser device which can contain one or more unit dosage forms containing a compound disclosed herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutically acceptable carrier can also be prepared, placed in an appropriate container, and labeled for treatment of an indicated condition.

### EXAMPLES

In order to provide a better understanding of the foregoing discussion, the following non-limiting examples are offered. Although the examples can be directed to specific embodiments, they are not to be viewed as limiting the invention in any specific respect.

Monomeric Cu(II) complexes derived from tripodal amines bearing 3,5-dimethyl-pyrazole moieties were shown to have high potency in vitro cytotoxicity against some cancer cells [16,17c]. The in vitro cytotoxicity of the synthesizedbridged-phenoxido-copper(II) complexes 1-6, the 1D polymeric complex 7, and their parent free ligands were tested against selected human cancer cell lines as well as MRC-5 normal cells. To clarify the mechanistic pathways of these complexes different biological approaches were conducted on the best performing cytotoxic complexes (4, 6 and 7) in order to understand their influence on the cellular cycle and cell modification of A2780.

### Synthetic Aspects of Ligands and Complexes

An effective general method was used to synthesize all the tripodal phenolate amine ligands in this work. A methanolic mixture containing the 2-,4-disubstituted phenol, the corresponding amine, one or 2 equivalent amounts of aqueous 37% HCHO and equivalent amount of triethylamine, Et₃N. Refluxing the solutions for 3-4 days resulted in the formation of the desired products in moderate to good yields (41-91%). Recrystallization of these compounds from ethyl acetate and activated charcoal afforded the pure products.

The interaction of a given tripodal-phenolate amine compound with Cu(NO₃)₂-3H₂O(H₂L³—H₂L⁷), or Cu(ClO₄)₂·6H₂O in case of HL¹ and HL² in MeOH and in the presence of two or one equivalent(s) of Et₃N afforded the corresponding shiny green-olive or green dinuclear doubly bridged-phenoxido-copper(II) complexes [Cu₂(L¹⁻²)₂](ClO₄)₂(1, 2), [Cu₂(L³-4)₂](3, 4) and [Cu₂(L⁵⁻⁶)₂(H₂O)]·2H₂O (5, 6) in moderate to high yields (56-92%). However, it should be pointed out that with the flexible H₂L⁷ ligand, an intense purple 1D polymeric, catena-[Cu(L⁷)](7) was isolated. Single crystals of X-ray quality were obtained either from dilute methanolic solutions and/or recrystallization from CH₃CN or acetone. The complexes dissolve in CH₃CN, slightly soluble in MeOH but more soluble in DMSO. The isolated compounds were characterized by CHN microanalyses, spectroscopic techniques, and conductivity measurements as well as by single crystal X-ray crystallography for the copper complexes.

### Characterization of the Compounds

All compounds were characterized by elemental microanalyses. The IR spectra of the tripodal phenolate amines and their corresponding copper complexes display general characteristic features. The ligands HL¹-H₂L⁷ show a shoulder or very weak broad band over the region 3230-3140 cm⁻¹, which could be assigned to the v(O—H) of the phenolic groups, however in some cases it was hard to detect this band. The weak to very weak series of bands seen over the frequency range 3050-2700 cm⁻¹ are due to v(C—H) of the aliphatic and aromatic groups, whereas the strong-moderate series of bands shown at 1620-1200 cm⁻¹ are most likely attributed to v(C—C, C═N, C—O). The ESI-MS of the ligands in MeOH, showed the 100% m/z base signal, that corresponds to the protonated parent ligand. The ¹H NMR (DMSO-d₆) spectra displayed peak positions: 6=8.7-7.1 (protons-py); 6.8-7.1 (protons-ph); 5.0-4.9 (phenolate-protons); 3.8-3.7 (CH₂-py); 3.5-3.3 (CH₂-ph); 2.5-2.1 ppm (CH₃-ph). However, in some cases the phenolate proton bands were not seen even up to 256 scans, most likely due to their low solubility, but it was detected in H₂L³ and H₂L⁶ compounds.

The IR spectra of the complexes 1-7 exhibited similar spectral pattern as that observed in their parent ligands, except the disappearance of the phenolic v(O—H) upon complexation. In addition, the two perchlorate complexes 1 and 2 displayed very strong band at 1079 and 1076 cm¹, respectively due to vas(O—Cl) of the perchlorate counter ions. Also, the O—H stretching frequency of the water of crystallization, v(H—O) in complexes 5 and 6 were detected over the 3320-3400 cm⁻¹ region.

To gain an idea about the thermal stabilities of the complexes, the thermogravimetric (TG) and differential scanning calorimetry (DSC) analyses were performed for representative samples of the complexes [Cu₂(L³)₂](3), [Cu₂(L⁵)₂(H₂O)]·2H₂O (5) and catena-[Cu(L⁷)](7) over the temperature range 23 0-900° C. and graphs are represented in FIG. S8. The thermal decomposition of 3 revealed four major steps at 241.3, 355.4-403.6, 441.9 and 573.0° C. accompanied by exothermic effects corresponding to weight loss 7.77% (C₅H₄N, calcd: 8.15%), 12.26% (C₇HsN₂, calcd: 12.55%), 15.66% (C₈H₇ClO, calcd: 16.14%) and 58.92% (C₂₈H₂₀C₁₄O₄, calcd: 58.71%), respectively. Complex 5 showed a weight loss accompanied by an endothermic-effect with a minimum at 72.3° C., which may be attributed to the elimination of the two water molecules of crystallization (weight loss calcd./exp.: 3.6%/4.4%), in addition to two more steps associated with exothermic effects for the loss of the fragments C₁₈H₄₀N₄ of the aliphatic amines and coordinated water (weight loss: exp./calcd:32.31/32.98), and phenolates C₃₂H₃₂O₄(weight loss: exp./calcd: 48.72/47.95). Complex 7 showed similar decomposition pattern to 5 with fragments due to the phenolates C₁₁H₄₀O (weight loss: exp./calcd: 31.59/31.43) and C₁₄H₂₀NO (weight loss: exp./calcd: 42.74/42.29) as well as the amino fragment C₄H₁₀N (weight loss: exp./calcd: 13.08/13.97).

The ESI-MS measured in CH₃CN of the complexes 3-7 was consistent with the base signal corresponding to the release of the protonated parent organic ligand H₂L³⁻⁷ with m/z=[H₂L³-⁷+H]⁺, whereas in the cationic complexes 1 and 2, the base peak was fully consistent with the cationic monomeric species with m/z=[Cu(L^{1,2})]⁺.

Whenever, the solubility permits, the molar conductivities, ∇_{M} of the complexes 5-7, measured in CH₃CN or DMSO gave ∇M values<5 0 Ω⁻¹ cm² mol⁻¹, which are in full agreement with the non-electrolytic nature of the complexes as predicted by their molecular formulas [31]. The measured ∇_{M} values of the complexes [Cu₂(L^{1,2})₂](ClO₄)₂(1 and 2) obtained in DMSO, were 282 and 278 Ω⁻¹ cm² mol⁻¹, respectively. These values are consistent with the 1:2 electrolytic behavior of the compounds ([Cu₂(L¹⁻²)₂]2'⁰+2 Cl₄⁻) [31,32].

In general, the UV-VIS spectra of the complexes, measured in DMSO and/or CH₃CN at room temperature, displayed a strong absorption band and a single broad/shoulder over the wavelength ranges of 410-500 and 620-710 nm, respectively. The former high energy band can be assigned to the bridged phenoxido charge transfer transition (L-O→Cu^{II} LMCT) [5,33], whereas the second low intense broad band, which is occasionally associated with another weaker broad band around 850-890 nm is attributed to d-d transition in five-coordinate Cu(II) complexes and more consistent with a distorted square pyramidal geometry (SP) around the central Cu(II) ion [16,34,35]. These geometrical assignments in DMSO, CH₃CN or acetone solution were also retained in the solid state and agree with structures determined by X-ray diffraction (see next section). The Amax values in the 620-710 nm region can be used as a criterion to give an idea about the ligand field strength of the tripodal phenolate ligands used in this study. It increases in the order: H₂L⁶<H₂L⁷<H₂L⁵<HL^{1.2}≈H₂L^{3.4}. The broadening and very close location of these bands in HL^{1,2} and H₂L^{3,4} series did not allow accurate prediction of their ligand field strengths. However, the results for the H₂L^{5,6} ligands, where both incorporated N,N-di-isopropyl groups at the terminal coordinated ethylenic moieties, are interesting and completely coincidence with previous observations that increasing the steric hindrance on the coordinated donor atoms (H₂L⁶) tend to decrease their ligand field strength as these were the case in complexes 5 and 6 [36]. The decrease of the ligand field of H₂L⁶ compared to H₂L⁵ may result from the presence of the electronegative chlorine atoms in H₂L⁶, which reduces the electron density on the coordinated alkyl N-atom and hence weakens its ligand field. In complex 7 no chelate ring was formed with the terminal N, N-dimethylaminopropylenic moiety, but instead the dianion (L⁷)₂-was bridging the Cu²⁺ centers through its two nitrogen atoms of the propyl groups (see X-ray structure of complex 7). The solution spectra of the complexes 1-7 in these media did not show any appreciable change over a period of one week, which indeed reflects their high stability in these media.

### Description of the Structures

(Cu₂(L′)₂](ClO₄)₂(1)

Title compound 1 consists of dinuclear [Cu₂(L¹)₂]2'⁰ complex cations and perchlorate counter anions. The phenolato oxygen atoms O1 and O1′ of two (Li¹ anions are bridging the two Cu(II) metal centers to form four-membered rings [Cu1-O1′'² 1.925(2), Cu1-O132 2.215(2), Cu1 . . . Cu1′=3.098 A, O1—Cu1-O1′=83.37(10), Cu1-O1—Cu1′=96.63(10)° ](FIG. 2). The Cu1 center has distorted 5+1 geometry with the four terminal sites occupied by the amine N1, pyridyl N2, N3 atoms as well as the methoxy O2′ atom. The Cu1-N bond distances are 1.990(3), 2.011 (3), and 2.022(3) Å, whereas Cu1O-2′ forms a semi-coordinative bond of 2.791(3) Å.

(Cu₂(L²)₂](ClO₄)₂(2)

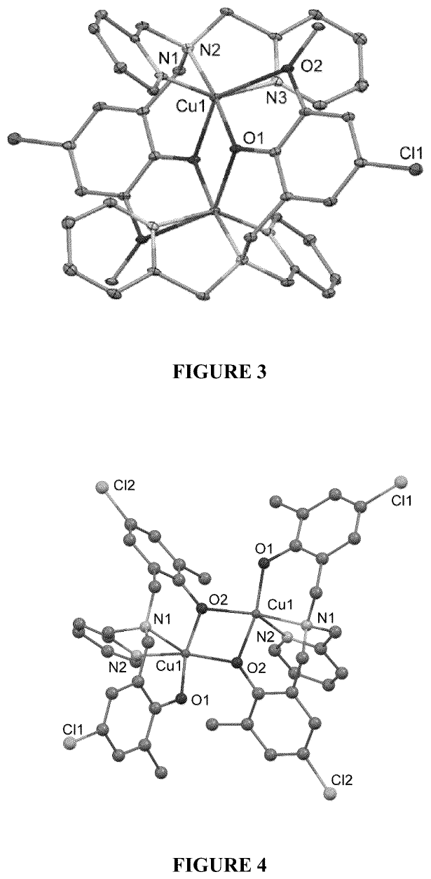

Compound 2 consists of centrosymmetric dinuclear [Cu₂(L²)₂]²⁺ complex cations and perchlorate counter anions. The phenolato oxygen atoms O1 and O1′ of two (L²)⁻ anions are bridging the two Cu(II) metal centers to form four-membered rings [Cu1-O1==1.936(3), Cu1-O1′=2.213(3), Cu1′-Cu1′²3.130 Å, O1—Cu1-O1′ '² 82.28(14), Cu1-O1—Cu1′ '² 97.72(14)° ](FIG. 3). The Cu1 center has distorted 5+1 geometry with the four terminal sites occupied by the amine N1′, pyridyl N2′, NY atoms as well as the methoxy O2 atom. The Cu1-N bond distances are 1.979(4), 2.017(4) and 2.019(4) Å, whereas Cu1-O2 forms a semi-coordinative bond of 2.785(4) Å.

[Cu₂(L³)₂](3)

Title compound 3 consists of dinuclear [Cu₂(L³)₂] neutral complex units. The phenolato oxygen atoms O2 and O2′ of two (L³)₂-anions are bridging the two Cu(II) metal centers to form four-membered rings [Cu1-O2=1.948(2), Cu1-O2′1.9900(18), Cu1′-Cu1′=3.0915(7) Å, O2-Cu1-O2′ '² 74.40(9), Cu1O-2-Cu1′ '² 103.44(9)° ](FIG. 4). The Cu1 center is penta-coordinated with a further three terminal sites occupied by the amine N1, pyridyl N2 atoms as well as the phenolate O1 atom. The CuN₂O₃ chromophore may be described as distorted square pyramid (SP) with a τ-value of 0.23 (τ=0 for ideal SP geometry and τ=1 for ideal TBP) [37]. The apical position is occupied by N2[Cu1-N2=2.310 (3) Å]. The remaining two basal bond lengths are Cu1-O1=1.877(2) and Cu1-N1=2.085(2) Å, and the trans-basal bond angles are O1—Cu1-02=166.92(8) and O2′—Cu1-N1=153.07(9).

### [Cu₂(L⁴)₂](4)

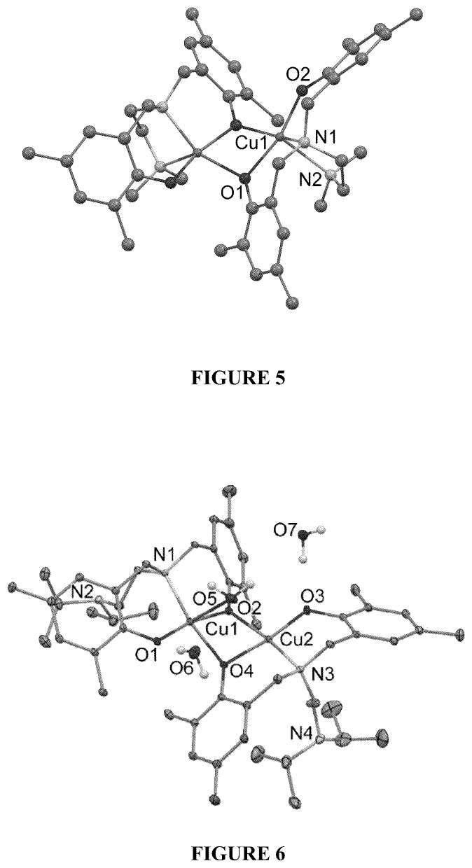

The title compound 4 consists of dinuclear [Cu₂(L⁴)₂] neutral complex units. The phenolato oxygen atoms O1 and O1′ of two (L⁴)₂-anions are bridging the two Cu(II) metal centers to form four-membered rings [Cu1-O1=1.9650(13), Cu1-O1′=1.9851(13), Cu1′-Cu1′=3.0064 (5) Å, O1—Cu1-O1′=75.40(6), Cu1-O1—Cu1′=99.12(6)° ](FIG. 5). The Cu1 center is penta-coordinated with further three terminal sites occupied by the amine N1 and N2 atoms as well as the phenolate O2 atom. The CuN2O3 chromophore may be described as distorted square pyramid (SP) with a τ-value of 0.24 [37]. The apical position is occupied by N2 [Cu1-N2=2.3580(17) Å]. The remaining two basal bond lengths are Cu1-02=1.8920(14) and Cu1-N1=2.0612(17) Å, and the trans-basal bond angles are O1—Cu1-02=165.25(6) and O1′-Cu1-N1=150.97(6).

[Cu₂(L⁵)₂(H₂O)]·2H₂O (5)

Compound 5 consists of the dinuclear [Cu₂(L⁵)₂(H₂O)] neutral complex units and two lattice water molecules per dinuclear complex unit. The phenolato oxygen atoms O2 and O4 of two (L⁵)²—anions are bridging the two Cu(II) metal centers to form four-membered rings [Cu1-O2=1.9376(17), Cu1-04 1.9956(18), Cu2-O2 1.9722(17), Cu2-04 1.9327(17), Cu1′—Cu2=2.9410(4) Å, O2-Cu1-04=74.78 (7), O2-Cu2−04=75.43(7), Cu1O-2-Cu2=97.56(7), Cu1-04—Cu2=96.93(7)° ](FIG. 6). The Cu1 center is penta-coordinated with three terminal sites occupied by the amine N1, phenolate O1 and aqua O5 atom. The CuO4N chromophore may be described as square pyramid (SP) with a τ-Cu1 value of 0.013 [37]. The apical position is occupied by aqua O5 with semi-coordinative Cu1-05 bond length of 2.512(2) Å. The remaining two basal bond lengths are Cu1-O1=1.8919(17) and Cu1-N1=2.026(2) Å, and the trans-basal bond angles are O1—Cu1-02=169.06(8) and O4—Cu1-N1=168.27(7°). Coordination number four around Cu2 is completed by amine N3 and phenolate O3 atoms [Cu2−03=1.8987(17), Cu2-N3=2.0293(19) Å]. The CuO3N chromophore may be described as intermediate between tetrahedral and square planar geometry with O3-Cu2-O4 and O2-Cu2-N3 bond angles of 169.72(8) and 166.19(8°), respectively. Due to the steric hindrance of the di-isopropyl groups on N2 and N4 atoms of the alkyl arms of the tripod dianion ligands, (L⁵)²−are not coordinated to the metal centers.

[Cu₂(L⁶)₂(H₂O)]·2H₂O (6)

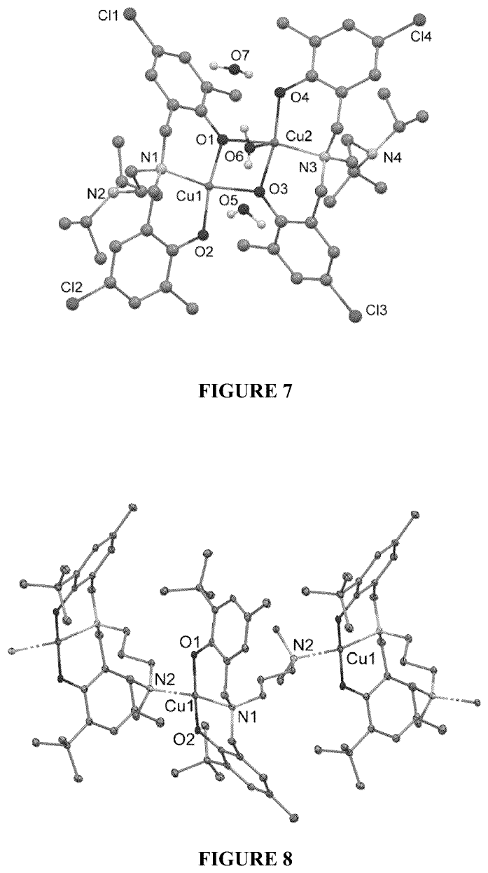

Title compound 6 consists of dinuclear [Cu₂(L⁶)₂(H₂O)] neutral complex units and two lattice water molecules per dinuclear complex unit. The phenolato oxygen atoms O1 and O3 of two (L⁶)²⁻ anions are bridging the two Cu(II) metal centers to form four-membered rings [Cu1-O1=1.9282(6), Cu1-O3=1.964(2), Cu2−01=1.9981(7), Cu2−O3==1.935(2), Cu1′—Cu2=2.9331 Å, O1—Cu1-O3=75.56(6), O1—Cu2−O3=74.61(6), Cu1-O1—Cu2=96.70(9), Cu1-O3-Cu2=97.58(9)° ](FIG. 7). The Cu2 center is penta-coordinated with further three terminal sites occupied by the amine N₃, phenolate O4 and aqua O6 atom. The CuO4N chromophore may be described as square pyramid (SP) with a r —Cu2value of 0.01 [35]. The apical position is occupied by aqua O6 with semi-coordinative Cu2-O6 bond length of 2.538(3) Å. The remaining two basal bond lengths are Cu2−04=1.893(2) and Cu2-N3=2.026(3) Å, and the trans-basal bond angles are O3-Cu2−04=168.30 (7) and O1—Cu2-N3=168.95(9°). Coordination number four around Cu1 is completed by amine N2 and phenolate O2 atoms [Cu1-02=1.985(2), Cu1-N1=2.0219(7) Å]. The CuO3N chromophore may be described as intermediate between tetrahedral and square planar geometry with O1—Cu1-O2 and O3-Cu1-N1 bond angles of 169.09(7) and 166.37(6°), respectively. Due to the steric hindrance imposed by the di-isopropyl groups at N2 and N4 atoms of the ethylenic arms of the dianion (L⁶)²⁻ ligands are not coordinated to the metal centers.

### Catena-[Cu(μ-L⁷)](7)

In contrast to the previous dinuclear complexes 1-6, the title complex 7 consists of mononuclear [Cu(L⁷)] units, which are linked to polymeric chains oriented along the c-axis of the monoclinic unit cell (FIG. 8). Each metal center is four-coordinated by 01, 02, N1 of same (L⁷)²⁻ ligand, as well as N2′ of N,N-dimethylpropyl arm of neighboring (L⁷)²⁻ tripod dianion ligand, to generate the 1D polymeric chain system, with intra-chain Cu . . . Cu separation of 7.269 Å. The Cu—O/N bond distances are: Cu1-O1=1.896(3), Cu1-02=1.894(3), Cu1-N1=2.034 (4), Cu1-N2′=2.091(4) Å. The CuN2O2 chromophore may be described as intermediate between tetrahedral and square planar geometry with O1—Cu—O2 and N1—Cu1-N2′ bond angles of 164.32(13) and 162.92(14°), respectively.

### In Vitro Cytotoxicity Against Human Cancer Cell Lines

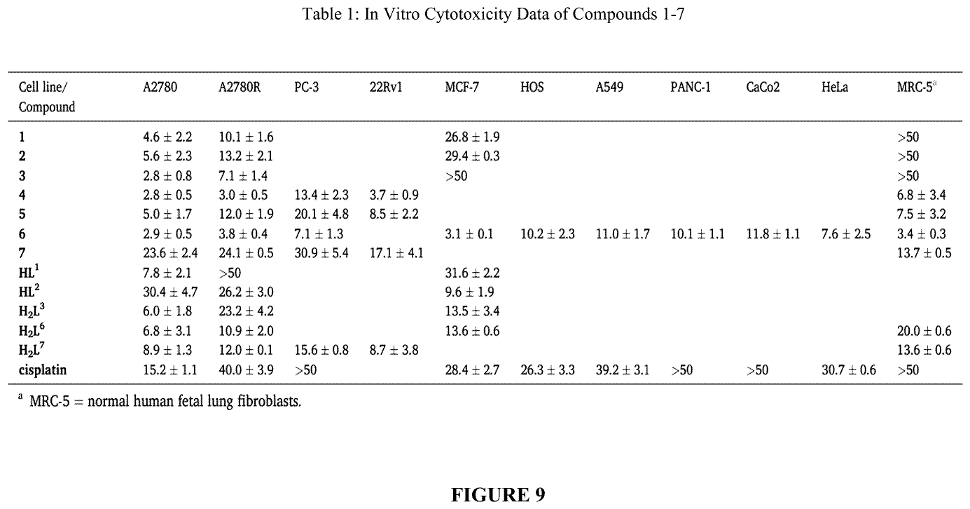

To reveal the in vitro cytotoxicity of the studied compounds against selected human cancer cell lines, the cytotoxicity of the copper(II) complexes 1-7, selected ligands and a reference drug cisplatin was determined by the MTT method against a panel of 2 cancer cell lines (A2780, A2780R). Based on the optimistic findings regarding cytotoxicity, the selected compounds were also studied on another three human cancer cells (PC-3, 22Rv1 and MCF-7). In the case of best performing complex 6 and a reference drug cisplatin, the panel of cancer cell lines has been extended to another five ones (HOS, A549, PANC-1, CaCo2, and HeLa). The results of cell-viability studies are summarized in the table shown in FIG. 9.

Both the selected free ligands and the corresponding copper(II) complexes showed in vitro cytotoxicity against the human cancer cells as well as healthy MRC-5 cells. As for the A2780 cell line, the cytotoxicity of complexes 1-7 was slightly higher or comparable with that of the corresponding free ligands, except for complex 2 whose cytotoxicity exceeded more than 5-times the cytotoxicity of the free H₂L² ligand, and for complex 7 whose cytotoxicity was ca 2.6-times lower than that of the free H₂L⁷ ligand, rendering the complex 7 a least active from the group. The relatively high antiproliferative effects of the tested ligands can be attributed to their iron and copper chelating capacities in cellulo, as this mechanism is one of the recognized approaches of anticancer drug design and polyamine residue can be considered one of the main phar-macophores [38, 39]. Regarding the cisplatin resistant A2780R cell line, the situation was very similar to A2780, while the most effective were the complexes 1, 3 and 6. Thus, the complexes 1-6 can be considered as the most promising ones based on the results following from these data obtained on A2780 and A2780R cell lines. Following the facts mentioned above and summarized in Table 1 of FIG. 9, it was decided to test the selected compounds on 3 more cancer cell lines (i.e., PC-3, MCF-7 and 22Rv1) with the aim to select the best performing complexes for consequent studies. The complex 6 has to be found as the most promising one and thus, its cytotoxicity was extended to include other 5 more-cancer cell lines (HOS, A549, PANC-1, CaCo2, and HeLa). The obtained results revealed that the complex 6 can be considered as a significant cytotoxic agent with better cytotoxicity than cisplatin on all the human cancer cell lines. The cytotoxicity of complex 6 against A2780 cells was practically independent from the intracellular levels of copper as shown by the cell-uptake results (see next section 2.5).

### Cellular Uptake of Complex 6 in the A2780 Cells

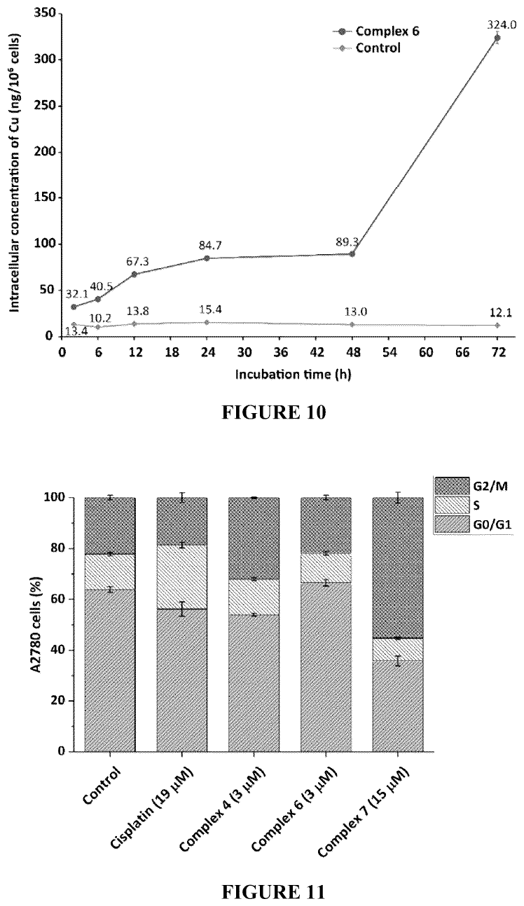

The cellular uptake of the best performing complex 6 (based on in vitro cytotoxicity results) was determined as time-dependent changes in intracellular copper content in the A2780 cells. Complex 6 was incubated with A2780 cells at 3 M concentration (the value of ic₅₀ determined in cytotoxicity studies) for 2, 6, 12, 24, 48 and 72 h, the samples were mineralized, and the copper content was determined by the ICP-MS method. In the untreated control cells, the copper intracellular levels were relatively stable in the range of 10.2-15.4 ng/10⁶ cells. In contrast, the cells treated with complex 6 showed gradual increase in intracellular copper levels up to 48 h (at 89.3 ng/10⁶ cells) with very rapid increase between 48 h and 72 h of incubation up to ca. 25-fold of the initial values. This result indicates that complex 6 possess a high ability to traverse cell membranes of A2780 cells and the high concentration can correlate with its significant cytotoxicity against this cancer cell line. The concrete values of copper intracellular levels in dependence of increasing time are shown in FIG. 10.

### Time Dependence of in Vitro Cytotoxicity Against the A2780 Cells

With the aim to deeper describe the antiproliferative effect of the best performing complex 6 0n the A2780 cell line, the time-dependent in vitro cytotoxicity (with the incubation times 24, 48 and 72 h) was determined as shown in Table 2).

TABLE 2
Time-dependent in vitro cytotoxicity of complex
6 and cisplatin on the A2780 cell line

| | Incubation time | | |
|---|---|---|---|
| Compounds | 24 h | 48 h | 72 h |
| Compound 6 | 3.6 ± 0.4 | 3.4 ± 0.4 | 3.1 ± 0.3 |
| Cisplatin | 18.0 ± 3.7 | 12.4 ± 1.8 | 5.8 ± 1.8 |

As it can be seen Table 2, while the cytotoxicity of complex 6 remains nearly constant within the time-period, the cytotoxicity of cisplatin is continuously decreasing within the time-period of 24-72 h. Thus, it may be concluded that nearly constant cytotoxicity of complex 6 in time has no relation to the increase in intracellular copper levels in A27890 cells thorough 72 h of incubation.

### Cell Cycle Modification of the A2780 Cells

The most cytotoxic complexes 4 and 6, and the least effective complex 7 as well as the reference drug cisplatin were chosen for flow cytometry studies with the aim to find out how can their presence influence cellular cycle of the A2780 cells. Surprisingly, the effects of the studied complexes were significantly different as shown in FIG. 11. The dimeric complex 6 applied at the 3 M concentration caused only a small increase in the portion of cells in GO/G1 cell phase. On the other hand, complex 4 and dominantly complex 7 caused significant increase in the number of cells arrested in G2/M phase, while the portion of cells in GO/G1 cells (cells which were not involved in the cell division) decreased accordingly. Similar cellular effects were described for a number of copper(II) complexes previously for a number of copper(II) complexes involving various ligands [40-43.] The effect of complexes 4, 6 and 7 0n A2780 cells division was very different from the reference cisplatin, causing the significant arrest of cells in S phase due to covalent modification of DNA. This indicates that the mechanism of action in these complexes is different from that of cisplatin and moreover, the mechanisms among the three complexes differ from each other as well (FIG. 11).

### Cell Death Induction in the A2780 Cells

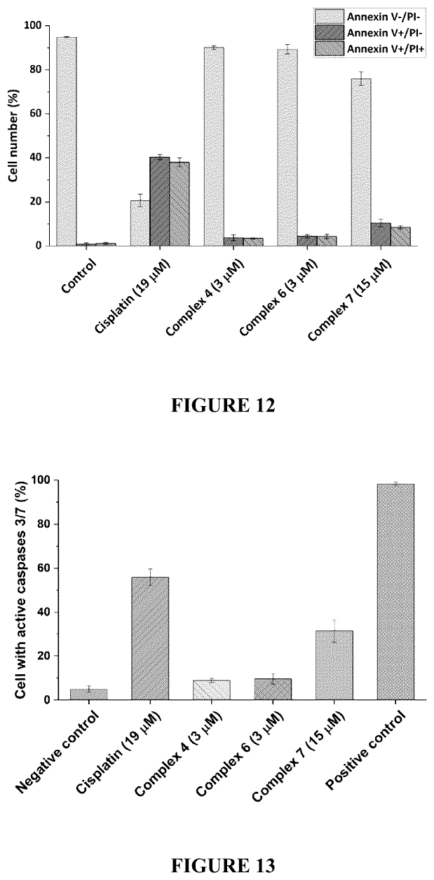

The induction of cell death in A2780 cancer cells was monitored by flow cytometry. Three types of cells were identified by incubation with Annexin V antibody and propidium iodide (PI). The normal cells were Annexin V and PI negative. The cells in early stages of apoptosis showed positivity towards the Annexin V detection, while the propidium iodide detection was negative because no intracellular interaction of PI with DNA of A2780 cells occurred (FIGURE 12). The cells in late stages of apoptosis and remnants of necrotic cells showed both reactions as positive. Although the complexes 4, 6 and 7, and a reference drug cisplatin, were applied at the concentration causing the 50% decrease in proliferation of A2780 cells, the most intensive induction of cell death processes (apoptosis and necrosis) were observed for cisplatin. The studied complexes caused far less evident induction of apoptosis with increasing activity in the order 4<6<7 as can be seen from FIG. 12.

To assess the participation of apoptosis on the cell death induced by the studied complexes, the activity of effector caspases 3/7 in A2780 cells incubated with the tested compounds was determined by flow cytometry. The results are shown in FIG. 13. Although complexes 4 and 6 proved to be the most cytotoxic ones, their effect on effector caspases 3/7 activation was minimal. This unequivocally means that the cell death they induce proceeds via a mechanism independent from caspases. On the other hand, the reference drug cisplatin and complex 7 caused significant activation of caspases 3/7 in A2780 cells after 24 h incubation, thus indicating that a main mechanism of cell death in cells interacting with these agents is apoptosis.

### Autophagy Induction

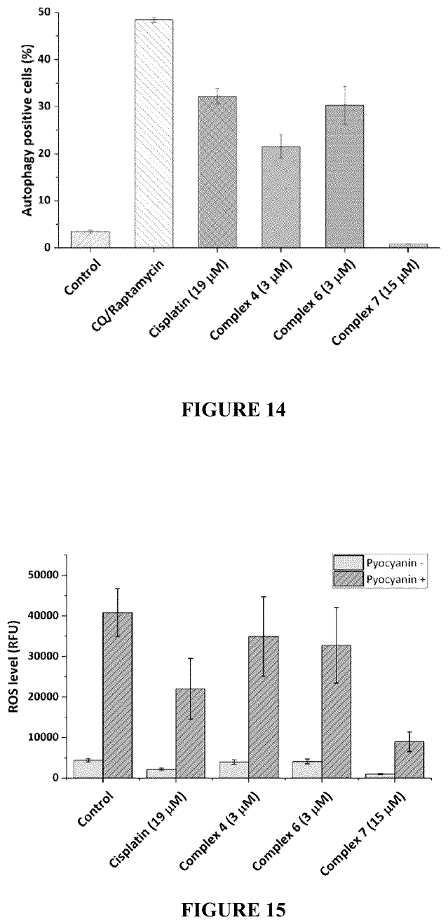

To further explore the mechanisms involved in the cellular death of A2780 cells induced by the complexes, the autophagy progression was monitored in the cells using a specific flow cytometry-based kit. The autophagy signaling caused by the incubation with the tested compounds was compared to the untreated control group of cells and positive control group, which included cells treated with the chloroquine/rapamycin (CQ/Rapamycin) mixture inducing strong autophagic response in cells. Only complexes 4 and 6, together with cisplatin showed a significant activation of autophagy in the A2780 cells (FIG. 14). Complex 7 had no effect on autophagy in A2780 cells. These results shed a new light on the previous findings about insignificant role of caspases activation caused by complexes 4 and 6, as their effect on cells' death in A2780 cells might be associated with the types of cell death other than apoptosis. In fact, autophagy might have a dual role in cancer as it can help the cancer cells to overcome metabolic stress (hypoxia, lack of nutrients) and suppress tumor progression through degradation of oncogenic proteins [44]. Many anticancer agents, including the copper(II) complexes [45], which mechanism of action includes different cellular processes, such as inhibition of mTORC1, proteasome inhibition, inhibition of histone deacetylases or topoisomerases [44,45], induce autophagy. On the other hand, the apoptosis is evidently a main route of cell death in the A2780 cells caused by complex 7.

### Induction of ROS/Superoxide Production

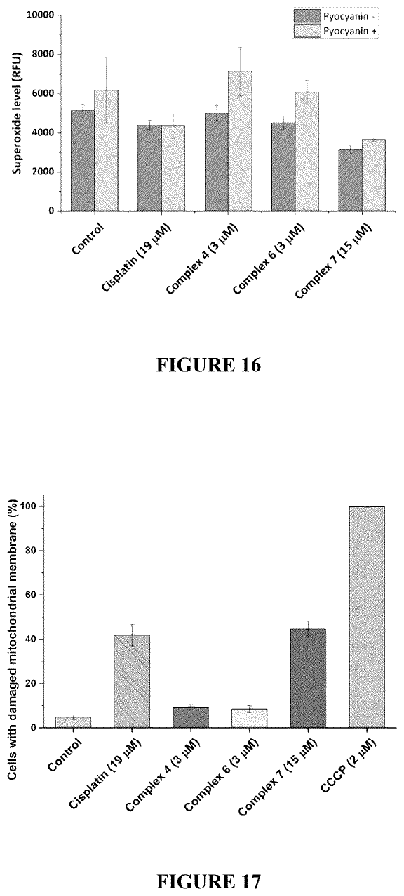

The effects of the tested compounds on the ROS production in A2780 cells and on the progression of the oxidative stress caused by the known pro-oxidant agent (pyocyanin) were observed by means of flow cytometry using a specific kit of intracellular ROS, and superoxide detection, respectively. In comparison with the untreated cells (control), none of the tested complexes 4, 6, and 7 nor cisplatin showed any significant abilities to promote the oxidative stress by themselves (FIG. 15). On the other hand, in the case of systems with the progressing oxidative stress caused by the pretreatment with pyocyanin, complexes 4, 6 and cisplatin showed only slight antioxidative effects, while complex 7 was able to significantly attenuate the oxidative stress in A2780 cells (FIG. 16). Approximately the same pattern was identified also in case of superoxide production (FIG. 16). The obtained results indicate that the mechanism of action of the tested copper(II) complexes is unlikely to be ascribed to DNA damage caused by ROS overproduction [40] and they are likely to target other cellular targets.

### Mitochondrial Membrane Disruption

In order to get more insight about the mechanisms involved in the induction of cell death in the A2780 cells treated with the complexes 4, 6, and 7, the detection of mitochondrial membrane potential in the cells was performed by means of flow cytometric analysis using a specific kit. Unsurprisingly, based on the results of caspase 3/7 activation, the impairment of the mitochondrial membrane function/permeability was caused by complex 7 and cisplatin only, as shown in FIG. 17. Both these agents induced the apoptosis via the intrinsic route of apoptosis activation, characterized by the efflux of cytochrome c from the damaged mitochondria to the cytoplasm of the cells, followed by the formation of apoptosis and activation of effector caspases 3/7 leading to the destruction of cell cytoskeleton and fragmentation of DNA. Complexes 4 and 6 caused only insignificant increase in the mitochondrial membrane damage, hence indicating that previous assumptions about their ability to induce the cell death processes independent from caspases (other than apoptosis) is the main mechanism of their cyto-toxicity in A2780 cells. The major suspect in this case is the induction of cuproptosis [46], a relatively newly discovered mechanism of cell death caused by copper, which is characterized by the high intracellular levels of copper, low levels of apoptosis, yet substantial antiproliferative effects on the target cells. The main molecular mechanism associated with cuproptosis is direct binding of copper to lipoylated components of the tricarboxylic acid (TCA) cycle in mitochondria, thus resulting in lipoylated protein aggregation and subsequent iron-sulfur cluster protein loss leading to proteotoxic stress and ultimately cell death [46].

### Toxicity Against Normal Cells

With the aim to find out, if the studied compounds are selective enough, the in vitro toxicity of the selected compounds to normal cells (MRC-5 human fibroblasts) was evaluated. The toxicity data showed reasonable selectivity of the complexes 1-3 (with selectivity index SI=IC50 (MRC-5)/IC50 (A2780) higher than 10), while the selectivity index in the case of the complexes 4-6 is negligible because the concentrations determined as the IC₅₀ values (cytotoxicity vs. toxicity) are in the same range.

A series of doubly bridged-phenoxido-copper(II) complexes of diverse geometrical arrangements around the central Cu(II) atoms [Cu₂(L¹⁻²)₂](ClO₄)₂(1, 2), [Cu₂(L³⁴)₂](3, 4) and [Cu₂(L⁵⁻⁶)₂(H₂O)]·2H₂O (5, 6) as well as 1D catena-[Cu(L⁷)](7), where HL¹⁻² and H₂L³⁻⁷ represent tripodal tetradentate pyridyl or aliphatic-amino groups based 2,4-disubstituted phenolates, has been synthesized and structurally characterized. The complexes showed significant stability in DMSO, CH₃CN and acetone for a period of more than one week without exhibiting any sign of hydrolysis, decomposition nor geometrical changes. Moreover, they retained square pyramidal geometry in these media as structurally determined in some of the crystalline solids. The in vitro cytotoxicity results of the compounds 1-7 and the reference drug cisplatin tested against some human cancer cell lines and normal cells (MRC-5 human fibroblasts) revealed that complexes 1-6 were the most promising anticancer agents against A2780 and A2780R cell lines. Interestingly, in this case, compounds 1-6 were at least 3-6 times more cytotoxic in reference to cisplatin drug. In addition, complex 6 showed significant cytotoxicity against the cancer cells PC-3, MCF-7, HOS,

A549, PANC-1, CaCo2, and HeLa. However, high selectivity index (cytotoxicity vs. toxicity) SI=IC50 (MRC-5)/IC50 (A2780) in the range of 5-15 were obtained in compounds 1-3, whereas comparable or negligible SI values were found in complexes 4-6. To account for the observed anticancer activity to the molecular structures of the bridged-phenoxido compounds one can consider that in closely related ligands replacement of the chlorine atoms in 2 with a methyl group in 1 did not show pronounced increase in the anticancer activity against A2780 and A2780R cancer cells and similar trend was also found between 6 and 5 complexes (see Scheme 1 and Table 2). The influence of the N-alkyl substituents in the terminal arms such as replacing the di-isopropyl groups in 5 with di-methyl in 4 resulted in slight increase of the cyto-toxicity of the latter complex by a factor of 2. Similar result was seen upon replacing pyridyl arms in 2 with a phenolate group (3).

### Materials and Physical Measurements

2,4-Dimethylphenol, 2-chloro-4-methylphenol, 2-methoxy-4-methyl-phenol, 3-dimethyl-aminopropylamine, N,N-diisopropylethylenediamine, picolylamine and N,N-dipicolylamine were purchased from TCI-America, whereas 2-chloro-4-methoxyphenol, 2-tert-butyl-4-methylphenol and N, N-diethylethylenediamine were obtained from Alfa Aesar. All other chemicals were commercially available and used without further purification.

Electronic spectra were recorded using an Agilent 8453 HP diode array UV-Vis. spectrophotometer. Infrared spectra were recorded on Cary 630 (ATR-IR) spectrometer. ¹H NMR spectra were obtained at room temperature on a Varian 400 NMR spectrometer operating at 400 MHz (1H). ¹H NMR chemical shifts (δ) are reported in ppm and were referenced internally to residual solvent resonances (DMSO-d₆: δ_{H} '² 2.49 ppm). ESI-MS of organic compounds and their Cu(II) were measured in MeOH and CH3CN, respectively on LC-MS Varian Saturn 2200 Spectrometer. Conductivity measurements were performed using Mettler Toledo Seven Easy conductivity meter and calibrated by 1413 μS/cm conductivity standard. The molar conductivity of the complexes was determined from ∇M=(1.0×10³κ)/[Cu(II)], where x=specific conductance and [Cu(II)] is the molar concentration of the copper complex. Thermogravimetric (TG) and differential scanning calorimetry (DSC) analyses were performed for complexes 3, 5 and 7, as representative samples, using Netzsch STA 449 F1 Jupiter@apparatus, in the temperature range of 23° C.-900° C., in air atmosphere, and with the temperature gradient of 5° C./min. Elemental microanalyses were carried out by Atlantic Microlaboratory, Norcross, Georgia U.S.A.

### X-Ray Crystal Structure Analysis

The X-ray single-crystal data of complexes 1-7 was collected on a Bruker-AXS SMART APEX II CCD diffractometer at 100(2) K. The intensities were collected with Mo-Kα radiation (λ=0.71073 Å). Data processing, Lorentz-polarization and absorption corrections were performed using SAINT, APEX and the SADABS computer programs [47a,b]. The structures were solved by direct methods and refined by full-matrix least-squares methods on F2, using the SHELXTL program package [48a,b]. All non-hydrogen atoms were refined anisotropically. The hydrogen atoms were located from difference Fourier maps, assigned with isotropic displacement factors and included in the final refinement cycles by use of geometrical constraints. Molecular plots were performed with the MERCURY program [49a,b]. Deposition numbers: CCDC-2205904-CCDC—2205910 for compounds 1-7, respectively.

### Synthesis of the Organic Ligands

### 2-((Bis(pyridin-2-ylmethyl)amino)methyl)-6-methoxy-4-methylphenol (HL¹)

A mixture of 2-methoxy-4-methylphenol (1.38 g, 10 mmol), Et₃N (1.02 g, 10 mmol), aqueous 37% HCHO (0.814 g, 10 mmol) and dipicolylamine (2.00 g, 10 mmol) was dissolved in methanol (40 mL). The mixture was stirred and refluxed gently for 3 days, during which color turns golden-yellow. The solvent was reduced to half of its volume by rotary evaporator and allowed to stand at room temperature. The off-white precipitate was filtered and recrystallized from EtOAc and activated charcoal, this was followed by the addition of anhydrous MgSO4, which was then removed and upon standing at room temperature, the separated white precipitate was collected by filtration, washed with Et₂O and dried in air (yield: 3.20 g, 91.4%). Characterization: m.p. 121-123° C. Anal. Calcd for C₂₁H₂₃N₃O₂ (MM=349.426 g/mol): C, 72.18; H, 6.63; N, 12.03%. Found: C, 72.18; H, 6.79; N, 12.07%. IR bands (ATR, cm⁻¹): 3226 (w, b) v(O—H); 3052 (vw), 2999 (w), 2953 (w), 2836 (w) v(C—H); 1589 (s) v(C═N); 1569 (s), 1474, 1431 (s), 1363 (s), 1324 (m), 1294 (m), 1277 (vs) v(C═C, C—O); 1160 (s), 1121 (m), 1083 (vs), 1048 (m), 992 (s), 976 (s), 869 (m), 843 (s), 786 (s), 756 (vs). ESI-MS: m/z=350.187 (100%), calcd [HL+H]⁺=350.187.¹H NMR (DMSO-d₆, 400 MHz, 6 in ppm): 8.76, 8.52, 7.43, 7.27 (8H, d, m, m, d, 1:1:1:1, 2xpy); 6.67, 6.60 (2H, 2s, 1:1, ph); 3.76, 3.72 (4H, 1:1, 2xCH₂-py); 3.64 (3H, s, CH₃O-ph); 3.35 (2H, s, CH₂-ph); 2.19 (2H, s, CH₃-ph).

### 2-((Bis(pyridin-2-ylmethyl)amino)methyl)-4-chloro-6-methoxyphenol (HL²)

This compound was synthesized using a procedure similar to that described above for HL, except 4-chloro-4-methylphenol was used instead of 2,4-dimethylphenol (yield: 3.20 g, 86.5%). Characterization: m.p. 119-122° C. Anal. Calcd for C₂₀H₂₀C1N₃O₂(MM=369.845 g/mol): C, 64.95; H, 5.45; N, 11.36%. Found: C, 64.93; H, 5.52; N, 11.38%. IR bands (ATR, cm ¹): −3175 (w, b) v(O—H); (w, b) 3005 (vw), 2958 (vw), 2902 (vw), 2835 (vw) v(C—H); 1590 (m) v(C═N); 1569 (m), 1475 (s), 1465 (s), 1438 (s), 1362 (s), 1377 (s), 1362 (s), 1229 (vs) v(C═C, C—O); 1079 (s), 993 (s), 976 (s), 881 (m), 856 (m), 840 (m), 752 (vs). ESI-MS: m/z=370.1279, calcd [HL²⁺ H]⁺=370.1322.¹H NMR (DMSO-d₆, 400 MHz, 6 in ppm): 8.53, 8.52,7.77, 7.27 (8H, d, m, m, d, 1:1:1:1, 2py); 6.67, 6.73 (2H, 2s, 1:1, ph); 3.76, 3.73 (4H, 2s, 1:1, 2xCH₂-py); 3.34 (3H, s, CH₃O-ph); 2,19 (2H, s, CH₂-ph).

### 6,6′-(((Pyridin-2-ylmethyl)azanediyl)bis(methylene))bis(4-chloro-2-methylphenol) (H₂L³)

A mixture of 4-chloro-2-methylphenol (5.71 g, 40 mmol), 2-picolyl-amine (2.16 g, 20 mmol), Et₃N (4.05 g, 40 mmol) and aqueous 37% HCHO (3.30 g, 40 mmol) was dissolved in CH₃OH (60 mL). The mixture was stirred and refluxed gently for 3 days, during which color turns deep wine-red. Solvent was removed under reduced pressure using rotary evaporator and residue was extracted with CH₂Cl₂ (3×30 mL) and washed with H₂O (3×25 mL), and anhydrous MgSO₄ was added to the DCM solution. After filtration, the resulting solution was charged on a column containing silica gel. The desired product was collected upon elution with 5% MeOH/CH₂Cl₂ (by volume). The collected organic layer was further treated with anhydrous MgSO₄, filtration and evaporating solvent resulted in the formation of a white precipitate, which was collected by filtration and recrystallization from EtOAc afforded the pure desired product (yield: 5.62 g, 67.4%). Characterization of H₂L³: m. p. 166-168. Anal. Calcd for C₂₂H₂₂C₁₂N₂O₂ (MM=417.33 g/mol): C, 63.32; H, 5.31; N, 6.71%. Found: C, 63.36; H, 5.19; N, 6.82%. IR bands (ATR, cm⁻¹): 2920 (vw), 2813 (vw), 2700 (vw) v(C—H); 1599 (m) v(C═N); 1573 (m), 1471 (s), 1433 (s), 1370 (s), 1283 (s), 1223 (vs) v(C═C, C—O); 1091 (m), 1006 (m), 974 (m), 856 (s), 758 (vs), 738 (vs). ESI-MS: m/z=417.113 (100%) and 418.116 (29.17), calcd for [H₂L³]=417.114 and [H₂L³+H]⁺=418.10, respectively. ¹H NMR (DMSO-d₆, 400 MHz, 6 in ppm): 8.62, 7.86, 7.40, 7.37 (4H, d, m, m, d, 1:1:1:1, py); 7.06 (4H, 2s, 1:1, ph); 4.97 (2H, s, HO-phenol); 3.83, 3.71 (4H, s, CH₂-py); 1.91 (6H, s, 2×CH₃-ph); 2.19 (4H, s, 2×CH₂-ph).

### 6,6′-(((2-(Dimethylamino)ethyl)azanediyl)bis(methylene))bis(2,4-dimethylphenol) (H₂L⁴)

To a mixture containing 2,4-dimethylphenol (2.44 g, 20 mmol), Et₃N (2.04 g, 20 mmol), aqueous 37% HCHO (1.63 g, 20 mmol, dissolved in methanol (50 mL), N,N-dimethylethylenediamine (0.88 g, 10 mmol). The mixture was stirred and refluxed gently for 3 days. Evaporating the resulting solution under reduced pressure resulted in the formation of white precipitate, which was then filtered, washed with Et₂O, and air dried (yield: 2.25 g, 63.1%). C₂₂H₃₂N₂O₂ (MM=356.502 g/mol): C, 74.12H, 9.05; N, 7.86%. Found: C, 74.15; H, 9.06; N, 7.81%. Characterization: m.p. 167-170° C. ESI-MS: m/z=357.255, calcd [H₂L⁴+H]⁺=357.254.¹H NMR (DMSO-d₆, 400 MHz, 6 in ppm): 6.85, 6.67 (4H, 1:1, 2xph); 3.57 (4H, s, 2×CH₂-ph); 2.56 (4H, s, N—CH₂—CH₂—N); 2.31, 2.27 (12 H, s, 1:1, 4xCH₃-ph); 2.20 (6H, s, (CH₃)₂N).

### 6,6′-(((2-(Diisopropylamino)ethyl)azanediyl)bis(methylene))bis(2,4-dimethylphenol) (H₂L⁵)

This ligand was isolated as white crystalline compound, and was synthesized essentially as described above for H₂L⁴, except N,N-diisopropylethylenediamine (1.842 g, 10 mmol) was used instead of the corresponding N,N-diethylethylenediamine (yield: 3,52 g, 85.3%). Characterization: m.p. 124-127° C. Anal. Calcd for _{C6H40N202} (MM=412.61 g/mol): C, 75.68; H, 9.77; N, 6.79%. Found: C, 75.59; H, 9.87; N, 6.93%. IR bands (ATR, cm⁻¹): 3230 centered (w, b) v(O—H); 2964 (m), 2918 (m), 2808 (w), 2720 (vw) v(C—H); 1617 (w), 1471 (s), 1486 (vs), 1459 (s), 1365 (m), 1217 (vs) v(C═C, C—O); 1156 (m), 1125 (m), 1106 (m), 1057 (m), 994 (m), 860 (vs), 814 (m), 767 (m), 688 (m). ESI-MS: m/z=413.318 (100%), calcd [H₂L⁵+H]⁺=413.317.¹H NMR (DMSO-d₆, 400 MHz, 6 in ppm): 6.79, 6.74 (4H, s, 1:1, 2xph); 3.62 (4H, s, 2×CH₂-ph); 2.80 (2H, m, 2×CH-isp); 2.47, 2.36 (4H, m, 1:1, 2×N—(CH₂)₂—N); 2.13, 2.10 (12H, s, 4xCH₃-ph); 0.80 (12H, d, 1:1, 2×CH₃-isp).

### 6,6′-(((2-(Diisopropylamino)ethyl)azanediyl)bis(methylene))bis(4-chloro-2 methylphenol) (H₂L⁶)

A mixture of 4-chloro-2-methylphenol (2.86 g, 20 mmol), Et₃N (2.04 g, 20 mmol), aqueous 37% HCHO (1.63 g, 20 mmol) and N,N-diisopropylethylenediamine (1.44 g, 10 mmol) was dissolved in methanol (50 mL). The mixture was stirred and refluxed gently for 3 days, during which color turns dark yellow. Evaporating the resulting solution resulted in the formation of faint yellow crystallin compound, which was then filtered, washed with Et₂O, and air dried (yield: 3.52 g, 84.2%). Characterization: m.p. 120-124° C. Anal. Calcd for C₂₄H₃₄C₁₂N₂O₂ (MM=452.200 g/mol): C, 63.57; H, 7.56; N, 6.18%. Found: C, 63.71; H, 7.70; N, 6.25%. IR bands (ATR, cm⁻¹): −3140 (sh) v(O—H); 2971 (m), 2850 (w), 2811 (w) v(C—H); 1588 (m), 1467 (vs), 1371 (s), 1276 (s), 1225 (vs) v(C═C, C—O); 1128 (s), 1107 (m), 1054 (m), 984 (m), 857(vs), 811 (m), 741 (s). ESI-MS: m/z=453.2065 (100%), calcd [H₂L⁶+H]⁺=453.2076.¹H NMR (DMSO-d₆, 400 MHz, 6 in ppm): 7.05, 7.02 (4H, s, 1:1, 2xph); 4.84 (HO-phenol); 3.67 (4H, s, 2×CH₂-ph); 2.80 (2H, m, 2×CH-isp); 2.45 (4H, s, N—(CH₂)₂—N); 2.13 (6H, s, 2×CH₃-ph); 0.82 (12H, s, 2×CH₃-isp).

### 6,6′-(((3-(Dimethylamino)propyl)azanediyl)bis(methylene))bis(2-(tert-butyl)-4-methyl-phenol) (H₂L′)

A mixture of 2-tert-butyl-2-methylphenol (8.213 g, 50 mmol), Et₃N (5.06 g, 50 mmol), aqueous 37% HCHO (4.058 g, 50 mmol) and 3-dime-thylaminopropylamine (2.555 g, 25 mmol) was dissolved in methanol (60 mL). The mixture was stirred and refluxed gently for 4 days, during which color turns yellowish orange. Evaporating this solution resulted in the formation of white precipitate, which was then collected by filtration, washed with Et₂O, and air dried (yield: 4.97 g, 43.7%). No attempts were made to improve the yield. Characterization: m.p. 138-140° C. Anal. Calcd for C₂₉H₄₆N₂O₂ (MM=454.69 g/mol): C, 76.60; H,10.20; N, 6.16%. Found: C, 76.62; H, 10.17; N, 6.13%. IRbands (ATR, cm⁻¹): 2950 (s), 2909 (vw), 2883 (vw), 2829 (vw) v(C—H); 1592 (w), 1465 (s), 1442 (vs), 1230 (vs), 1202 (m) v(C═C, C—O); 1186 (s), 1164 (m), 1017 (m), 858 (s), 779 (m), 748 (s). ESI-MS: m/z=455.364 (100%), calcd [H₂L⁷+H]⁺=455.364.¹H NMR (DMSO-d₆, 400 MHz, 6 in ppm): 6.88, 6.75 (4H, s, 1:1 2xph); 3.55 (4H, s, 2×CH₂-ph); 2.43 (4H, m, 2× N—CH₂—C-prop); 2.16 (6H, s, 1:1, 2×CH₃-ph); 2.11 (6H, s, (CH₃)₂N); 1.65 (2H, m, C—CH₂—C); 1.33 (18H, s, 2x(CH₃)₃C).

Synthesis of copper(II) complexes

A general method was used to synthesize the copper(II) complexes: To a mixture containing the tripod phenolate ligand (H₂L³-H₂L⁷) (0.5 mmol) and Et₃N (0.50 mmol, 0.051 g) dissolved MeOH (20-30 mL), Cu(N₃)₂·3H₂O (0.5 mmol, 0.121 g) or Cu(ClO₄)₂·6H₂O (0.5 mmol, 0.185 g) was added and the resulting solution was heated for 5-10 min, filtered while hot through celite and allowed to crystallize ate room temperature. In case of HL¹ and HL² ligands, equimolar amounts of Et₃N and only Cu(ClO₄)₂-6H₂O were used. The precipitate was collected by filtration, washed with Et₂O and dried in air. Crystals suitable for X-ray analysis were obtained from dilute methanolic solutions or recrystallization from CH₃CN (acetone was used in complex 4).

### [Cu₂(L′)₂](ClO₄)₂(1)

Shiny olive-green crystals were separated after 2 h (yield: 155 mg, 60.6%) and X-ray quality crystals were obtained from dilute solution. Characterization: Anal. Calcd for C₄₂H₄₄Cl₂Cu₂N6O₁₂(MM=1022.830 g/mol): C, 49.32; H, 4.34; N, 8.22%. Found: C, 49.18; H, 4.72; N, 7.94%. IR bands (ATR, cm⁻¹): 3075 (vw), 2935 (vw), 2912 (vw) v(C—H); 1608 (m) v(C═N); 1572 (s), 1483 (vs), 1316 (m), 1256 (vs) v(C═C, C—O); 1079 (vs) v(O—Cl, ClO₄—); 1160 (m), 957 (m), 845 (m), 814 (m), 768 (s), 620 (s), 459 (m), 412 (m). ESI-MS (CH₃CN): m/z=411.096 (100%), calcd [Cu(L′)]⁺=411.101. UV-VIS: Amax in nm (mₐₓ, M⁻¹ cm⁻¹): in DMSO: 503 (939, b), −630 (sh), 870 (238, vb); in CH₃CN: 464 (410), 890 (309). ∇_{M} (CH₃CN)=282 Ω⁻¹ cm² mol⁻¹ and the same value was obtained in DMSO (average of two independent measurements).

### [Cu₂(L²)₂](ClO₄)₂(2)

Shiny light olive-green crystals were isolated in the following day (yield: 150 mg, 56.4%) and X-ray quality crystals were obtained from dilute solution. Anal. Calcd for C₄₀H₃₅Cl₄Cu₂N₆O₁₂ (MM=1063.667 g/mol): C, 45.17; H, 3.60; N, 7.90%. Found: C, 45.12; H, 3.66; N, 7.92%. IR bands (ATR, cm⁻¹): 3078 (vw), 2930 (vw), v(C—H); 1610 (m) v(C═N); 1471 (s), 1444 (m), 1316 (m), 1244 (vs) v(C═C, C—O); 1076 (vs) v(0—Cl, ClO₄); 959 (m), 859 (m), 792 (m), 620 (vs), 459 (m). UV-VIS in CH₃CN: kmax in nm (saturated): 449,625 (sh), 865 (vb). ∇_{M} (DMSO)=278 Ω⁻¹ cm² mol-¹ (average of two independent measurements).

### [Cu₂(L³)₂](3)

Shiny dark brownish-green crystals were isolated in the following day (yield: 190 mg, 75.2%.) and X-ray quality crystals were obtained from dilute solution. Characterization: Anal. Calcd for C₄₄H₄₀Cl₄Cu₂N₄O₄ (MM=957.717 g/mol): C, 55.18; H, 4.21; N, 5.85%. Found: C, 55.58; H, 4.37; N, 5.98%. IRbands (ATR, cm⁻¹): 2890 (vw), 2843 (vw) v(C—H); 1598 (m) v(C═N); 1462 (s), 1437(s), 1305 (s), 1241 (vs) v(C═C, C—O); 867 (m), 850 (m), 769 (vs), 750 (vs), 651 (m), 728 (m), 607 (m), 498 (m). ESI-MS (CH₃CN): m/z=417.133 (100%), calcd [H₂L³+H]⁺=417.114. UV-VIS in CH₃CN: λₘₐₓ in nm (saturated): 410 (b), −620 (w, sh).

### [Cu₂(L⁴)₂](4)

The crude solid which separated after 5 days from intense brownish-orange solution, was collected by filtration. Single brownish-green crystals suitable for X-ray analysis were obtained after crystallization from acetone (yield: 184 mg, 88.0%). Characterization: Anal. Calcd for C₄₄H₆₀Cu₂N₄O₄ (MM=836.064 g/mol): C, 63.21; H, 7.23; N, 6.70%. Found: C, 63.47; H, 7.26; N, 6.79%. IR bands (ATR, cm⁻¹): 2984 (vw), 2955 (m), 2960 (m), 2826 (m), 2792 (vw) v(C—H); 1609 (m), 1566 (vw), 1475 (vs), 1432 (s), 1391 (m), 1317 (s), 1248 (s) v(C═C, C—O); 1161 (s), 1101 (s), 1033 (m), 1017 (m), 871 (s), 857 (s), 800 (m), 775 (vs), 767 (vs). UV-VIS (saturated DMSO): max in nm: 430, 640, 733 (b).

### [Cu₂(L⁵)_(H₂O)]·2H₂O (5)

Shiny olive-green crystals of X-ray quality were obtained after 2 h (yield: 194 mg, 76.2%). Characterization: Anal. Calcd for C₅₂Hs₂Cu₂N₄O₇ (MM=1002.32 g/mol): C, 62.31; H, 8.25; N, 5.59%. Found: C, 62.18; H, 8.21; N, 5.62%. IR bands (ATR, cm⁻¹): 3400 (vw), v(0—H); 2959 (w), 2910 (m), 2856 (w) v(C—H); 1610 (w), 1472 (vs), 1384 (w), 1306 (s), 1251 (vs) v(C═C, C—O); 1160 (m), 857 (s), 806 (vs), 613 (m), 769 (vs), 750 (vs), 651 (m), 553 (m), 504 (s). ESI-MS (CH₃CN): m/z=413.316 (100%), calcd [H₂L⁵+H]⁺=413.317. UV-VIS: λₘₐₓ in nm (εₘₐₓ, M⁻¹ cm⁻¹): in CH₃CN: 439, 655 (690, b). ∇_{M} (CH₃CN)=2.3 Ω⁻¹ cm² mol⁻¹.

### [Cu₂(L⁶)₂(H₂O)]·2H₂O (6)

High quality dark olive-green needles were isolated in the following day (yield: 192 mg, 70.8%.). Characterization: Anal. Calcd for C₄₈H₇₀C₁₄Cu₂N₄O₇ (MM=1083.996 g/mol): C, 53.18; H, 6.51; N, 5.17%. Found: C, 53.23; H, 6.51; N, 5.21%. IR bands (ATR, cm⁻¹): 3319 (vw, b), v(O—H); 2961 (w), 2922 (vw), 2867 (w) v(C—H); 1613 (vw), 1459 (vs), 1435 (s), 1284 (m), 1241 (vs) v(C═C, C—O); 861 (m), 771 (vs), 652 (m), 611 (m), 462 (m). ESI-MS (CH₃CN): m/z=453.207 (100%), calcd [H₂L⁶+H]⁺=453.208. UV-VIS: amₐₓ in nm (8max, M⁻¹ cm⁻¹): in DMSO: 422 (1550), 708 (338, b); in CH₃CN (saturated): 408, 656 (b). ∇_{M} (DMSO)=5.0 Ω⁻¹ cm² mol⁻¹.

### Catena-[Cu(—L⁷)](7)

The crude dark brown precipitate, which was isolated in the following day (yield: 105 mg, 81.4%) was recrystallized from CH₃CN to afforded intense purple shiny crystalline compound. Characterization: Anal. Calcd for C₂₉H₄₄CuN₂O₂(MM=516.218 g/mol): C, 67.47; H, 8.59; N, 5.43%. Found: C, 67.55; H, 8.50; N, 5.45%. IR bands (ATR, cm⁻¹): 2947 (m), 2908 (m), 2858(m) v(C—H); 1608 (w), 1566 (vw), 1463 (vs), 1434 (vs), 1271 (vs), 1250 (s) v(C═C, C—0); 1008 (m), 923 (m), 853 (s), 812 (vs), 794 (m), 772 (m), 546 (s), 512 (m), 487 (m), 454 (m). ESI-MS (CH₃CN): m/z=455.363 (100%), calcd for [H₂L⁷+H]⁺=455.364. UV-VIS: λₘₐₓ in nm (εₘₐₓ, M⁻¹ cm⁻¹) in DMSO: 448 (699), 687 (337, b). ∇_{M} (DMSO)=2.3 Ω⁻¹ cm² mol⁻¹.

### Biological Activities Evaluation

### In Vitro Cell Viability Using MTT Test

The in vitro cytotoxicity of the prepared copper(II) complexes was determined by the standard MTT assay after the 24 h (and also 48 h and 72 h for complex 6 using A2780 cells) of treatment with the tested compounds (solutions in 0.1% DMF). The viability of cells was calculated based on the spectrophotometric detection of MTT-formazane at 570 nm using a microplate reader (Infinite M200 (Schoeller Instruments, Prague, Czech Republic) and compared with the untreated samples (0.1% DMF), and positive control (1% Triton X-100) respectively. The testing was performed on a panel of human cancer cell lines: ovarian carcinoma (A2780), cisplatin-resistant ovarian carcinoma (A2780R), prostate adenocarcinoma (PC-3), prostate carcinoma (22Rv1), breast adenocarcinoma (MCF-7), osteosarcoma (HOS), lung adenocarcinoma (A549), pancreatic epithelioid carcinoma (PANC-1), colorectal adeno-carcinoma (Caco-2), and cervical carcinoma (HeLa) obtained from ATCC collection of cell lines and cultivated according to producer's instructions. The reference normal cell line of human fetal fibroblasts (MRC-5) was obtained ibidem and maintained according to the producer's instructions. The half-maximal inhibitory concentrations (IC50) were calculated from dose-response curves by means of the GraphPad Prism 6 software (GraphPad Software, San Diego, USA).

### Cellular Uptake of 6 in A2780 Cells

The A2780 cells (10⁶ cells/well) were seeded in 6-well culture plates and incubated for 2, 6, 12, 24, 48, and 72 h with the solutions of complex 6 (at 3 M concentration) or cisplatin (at M concentration) prepared in 0.1% DMF. After incubation, the cells were washed twice with PBS and harvested by the trypsinization and pelletized by centrifugation. The isolated pellets were mineralized with 500 L of concentrated nitric acid for ICP-MS (65%, at 70° C., overnight). After that, the clear solutions were diluted by 4.5 mL of ultrapure water and the copper (or platinum in case of cisplatin) content was determined by ICP-MS method (ICP-MS spectrometer 7700x, Agilent).

### Cell Cycle Analysis

The A2780 cells (Sigma, 93112519-1VL) were cultivated according to the producer's instructions and seeded at the concentration of 10⁴ cells/well in 96-wells. The cells were treated with 3 μM solutions of the copper(II) complexes 4 0r 6, or 15 M solution of the complexes 7 0r 19 μM solution of cisplatin used as a reference standard. After 24 h, the cells were washed once with PBS (0.1 M, pH 7.4) and cell cycle analysis was performed according to the protocol based on BD Cycletest™ Plus DNA kit (Becton Dickinson, USA). Measurements were performed using BD FACSVerse flow cytometer (Becton Dickinson, USA) in three independent experiments, each done in duplicate, while at least 5×10³ events were recorded for each sample.

### Induction of Cell Death and Related Processes

The participation of the representative complexes 4, 6, 7, and cisplatin (applied at 3, 3, 15, and 19 M concentration, respectively) in the cellular processes involving in the cell death was evaluated using two methods in A2780 cells: i) detection of apoptosis using Annexin V-FITC apoptosis detection kit (Enzo Life Sciences, USA) and ii) activation of caspases 3/7 was determined by CellEvent™ Caspase-3/7 Green Flow Cytometry Assay Kit (Thermo Fisher Scientific, USA). Both assays were performed according to the manufacturers' protocols with one modification in caspase induction assay, which was the use of CellEvent™ Caspase-3/7 Green Detection Reagent only for detection of Caspase-3/7 activation. General procedure was as follows: 5×10³ cells were seeded per well in 24-well plate and after 24 h, the cells were treated either with the solutions of copper(II) complexes 4 (at 3 μM), 6 (at 3 μM), 7 (at 15 μM) or 19 M solution of cisplatin in 0.1% DMF. After the 24 h incubation period, cells were washed once with PBS (0.1 M, pH 7.4), detached by trypsinization (0.25% trypsin-EDTA (Gibco™), resuspended in 500 L of culture medium and divided into two separate samples. The first sample was used in the apoptosis assay and the second one was used to detect the activation of caspases 3/7 in A2780 cells. The samples were analyzed using the BD FACSVerse flow cytometer (Becton Dickinson, USA) in three separate experiments, while at least 10⁴ events were recorded for each sample prepared in duplicates.

Induction of autophagy in A2780 cells

The induction of autophagy by the selected complexes 4, 6, 7, and cisplatin applied at the 3, 3, 15, and 19 μM, concentration respectively, was evaluated using CYTO-ID® Autophagy Detection Kit 2.0 (ENZO, USA). The analyses were performed according to the manufacturer's protocol on A2780 cells pretreated for 24 h by the tested compounds. Briefly, the cells were trypsinized using 0.25% trypsin-EDTA solution (Gibco™) and the resulting suspensions were washed with PBS and adjusted to the final concentration of 10⁶ cells/mL. Suspensions were then mixed with an equal volume of CYTO-ID® Green stain solution, incubated for 30 min in the dark and finally washed with PBS. The samples were analyzed using the BD FACSVerse flow cytometer (Becton Dickinson, USA) in three separate experiments, while at least 10⁴ events were recorded for each sample prepared in duplicates. Cells incubated with chloroquine (10 iM) and rapamycin (0.5 iM) for 18 h were used as a positive control.

### Induction of Intracellular Production of ROS/Superoxide

The effect of the representative complexes 4, 6, 7, and cisplatin applied at 3, 3, 15, and 19 iM concentration, respectively, was evaluated on A2780 cells after 24 h of treatment. The oxidative stress analysis was performed using ROS-ID® Total ROS/Superoxide detection kit (Enzo Life Sciences, US) according to the manufacturer protocol. The samples were analyzed in triplicates on multimode microplate reader Infinite PRO M200 (Tecan, Switzerland). The control samples contained 500 iM pyocyanin.

### Mitochondrial Membrane Potential Analysis

The A2780 cells (5′ 10³ cells/well) were incubated with the selected complexes 4, 6, 7, and cisplatin applied at 3, 3, 15, and 19 M concentration, respectively for 24 h. The cells were washed with PBS, detached by trypsin treatment, isolated by centrifugation, and resuspended in 500 iL of culture medium. The cells were stained with MITO-ID® Membrane potential detection kit (Enzo Life Sciences, USA) according to the manufacturer protocol. The samples were measured in triplicates, while at least 10⁴ events were recorded using BD FACSVerse flow cytometer (Becton Dickinson, USA). The cells treated with 2 iM carbonyl cyanide 3-chlorophenylhydrazone (CCCP) were used as a positive control.

### Statistical Evaluations

The obtained results of biological activities were analyzed by one-way ANOVA followed by Fisher's LSD post-hoc test using Statistical 13 software (Tibco) and significant differences between the relevant sets of results were highlighted as follows: *p<0.05, **p<0.001, ***p<0.0001.

One of ordinary skill in the art will readily appreciate that alternative but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skills in the art to employ the present invention.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. It should also be appreciated that the numerical limits may be the values from the examples. Certain lower limits, upper limits and ranges appear in at least one claim below. All numerical values are “about” or “approximately” the indicated value, and consider experimental error and variations that would be expected by a person having ordinary skill in the art.

### REFERENCES

[1] Cancer Facts & Figures, American Cancer Society, 2022.
[2] a) T. C. Johnstone, K. Suntharalingam, S. J. Lippard, Chem. Rev. 9 (2016) 3436-3486, https://doi.org/10.1021/acs.chemrev.5b00597; b) T. C. Johnstone, G. Y. Park, S. J. Lippard, Anticancer Res. 34 (2014) 471-476.
[3] a) R. Oun, N. J. Wheate, Encyclopedia of Metalloproteins: Platinum Anticancer Drugs, DOI: https://doi.org/10.1007/978-1-4614-1533-6_525; b) C. Lange, P. J. Bednarski, Int. J. Mol. Sci. 19 (2018) 3183, https://doi.org/10.3390/ijms19103183; c) S. Dilruba, G. V. Kalayda, Cancer Chemother. Pharmacol. 77 (2016) 1103-1124; d) X. Wang, Z. Guo, Chem. Soc. Rev. 42 (2013) 202-222.
[4] a) V. Brabec, O. Hrabina, J. Kasparkov, Coord. Chem. Rev. 351 (2017) 2-31; b) R. Oun, Y. E. Moussa, N. J. Wheate, Dalton Trans. 19 (2018) 6645-6653; c) D.-W. Shen, L. M. Pouliot, M. D. Hall, M. M. Gottesman, Pharmacol. Rev. 64 (2012) 706-721, https://doi.org/10.1124/pr.111.005637.
[5] a) N. Sarin, F. Engel, F. Rothweiler, J. Cinatl, M. Michaelis, R. Frotschl, H. Fro′hlich, G. V. Kalayda, Int. J. Mol. Sci. 19 (2018) 767, https://doi.org/10.3390/ijms19030767; b) L. Galluzzi, I. Vitale, J. Michels, C. Brenner, G. Szabadkai, A. Harel-Bellan, M. Castedo, G. Kroemer, Cell Death Dis. 5 (2014), e1257, https://doi.org/10.1038/cddis.2013.428; c) D. Fink, S. Nebel, S. Aebi, H. Zheng, B. Cenni, A. Nehm.
[6] a) Å. Casini, A. Vessieres, S. M. Meier-Menches (Eds.), Metal-based Anticancer Agents, RSC Publication, 2019; b) J. C. Dabrowiak, Metals in Medicine, second ed., John Wiley; c) M. Frezza, S. Hindo, C. Di, A. Davenport, S. Schmitt, D. Tomco, Q. P. Dou, Curr. Pharmaceut. Des. 16 (2010) 1813-1825, https://doi.org/10.2174/138161210791209009.
[7] a) R. Paprocka, M. Wiese-Szadkowska, S. Janciauskiene, T. Kosmalski, M. Kulik, A. Helnin-Bas, Coord, Chem. Rev. 452(2022), 214307, https://doi.org/10.1016/j. ccr.2021.214307; b) D. A. Guk, 0.0. Krasnovskaya, E. K. Beloglazkina, Russ. Chem. Rev. 90 (2021) 1566-1623, https://doi.org/10.1070/RCR5016.
[8]R. Tabti, N. Tounsi, C. Gaiddon, E. Bentouhami, L. D′esaubry, Med. Chem. 7 (5) (2017) 875-879; b) S. Carlo, P. Maura, G. Valentina, P. Marina, T. Francesco, M. Cristina, Chem. Rev. 114 (2014) 815-862; c) M. Cristina, P. Maura, T. Francesco, S. Carlo, Med. Chem. 9 (2) (2009) 185-211.
[9]C. Molinaro, A. Martoriati, L. Pelinski, K. Caillia, Cancers 12 (2020) 2863, https://doi.org/10.3390/cancers12102863.
[10] a) M. Ghorbanpour, B. Soltani, A. Mota, J. J. Sardroodi, E. M. Aghdam, A. Shayanfar, O. Molavi, R. Mohammad-Rezaei, M. Ebadi-Nahari, C. J. Ziegler, Biometals 35 (2022) 1095-1111; b) Y.-Q. Gu, Y.-J. Zhong, M.-Q. Hu, H.-Q. Li, K. Yang, Q. Dong, H. Liang, Z.-F. Chen, Dalton Trans. 51(2022) 1968-1978; c) S. M. G. Leite, L. M. P. Lima, S. Gama, F. Mendes, M. Orio, I. Bento, A. Paulo, R. Delgado, O. Iranzo, Inorg. Chem. 55 (2016) 11801-11814; d) N. Ribeiro, S. Roy, N. Butenko, I. Cavaco, T. Pinheiro, I. Alho, F. Marques, F. Avecilla, J. C. Pessoa, I. Correia, J. Inorg. Biochem. 174 (2017) 63-75; e) S. Mignani, N. El Brahmi, L. Eloy, J. Poupon, V. Nicolas, A. Steinmetz, S. El Kazzouli, M. M. Bousmina, M. Blanchard-Desce, A.-M. Caminade, J.-P. Majoral, T. Cresteil, Eur. J. Med, Chem 132 (2017) 142-156.
[11]W. M. T. Q. de Medeiros, M. J. C. de Medeiros, E. M. Carvalho, J. A. de Lima, V. da S. Oliveira, A. C. F. de B.Pontes, F. O. N. da Silva, J. A. Ellena, H. A. de O. Rocha, E. H. S. de Sousa, D. de L. Pontes, RSC Adv. 8 (2018) 16873-16886.
[12]A. Sirbu, O. Palamarciuc, M. V. Babak, J. M. Lim, K. Ohui, E. A. Enyedy, S. Shova, a, P. Rapta, W. H. Ang, V. B. Arion, Dalton Trans. 46 (2017) 3833-3847.
[13]S. D. Kettenmann, Y. Nossol, F. R. Louka, J. R. Legrande, E. Marine, R. C. Fischer, F. A. Mautner, V. Hergl, N. Kulak, S. S. Massoud, INORGA 9 (2021) 12, https://doi. org/10.3390/inorganics9020012.
[14]R. Buchtik, Travnicek, J. Vanco, R. Herchel, Z. Dvor.
[15]M. Jopp, J. Becker, S. Becker, A. Miska, V. Gandin, C. Marzano, S. Schindler, Eur. J. Med. Chem. 132 (2017) 274-281.
[16]S. S. Massoud, F. R. Louka, M. T. Dial, J. F. Malek, R. C. Fischer, F. A. Mautner, J. Vanco, T. Malina, Z. Dvorak, Z. Travniceke, Dalton Trans. 50 (2021) 11521-11534, https://doi.org/10.1039/D1DT01724A.
[17] a) S. S. Massoud, F. R. Louka, G. T. Ducharme, R. C. Fischer, F. A. Mautner, J. Vanco, R. Herchel, Z. Dvorak, Z. Travnicek, J. Inorg. Biochem. 180 (2018) 39-46, https://doi.org/10.1016/j.jinorgbio.2017.11.023; b) S. S. Massoud, F. R. Louka, A. F. Tusa, N. E., N. E, R. C. Fischer, F. A. Mautner, J.ak, Z. Trek, New J. Chem. 43(2019) 6186-6196; c) R. Herchel, Z. Dvorak, Z. Travnicek, M. Mikuriya, F. R. Louka, F. A. Mautner, S. S. Massoud, Inorg. Chim. Acta. 451 (2016) 102-110, https://doi.org/10.1016/j. ica.2016.06.030.
[18] a) J.-J. Qua, P. Baib, W.—N. Liu, Z.-L. Liu, J.-F. Gong, J.-X. Wang, X. Zhu, B. Song, X.-Q. Hao, Eur. J. Med. Chem. 244 (2022),114859, https://doi.org/10.1016/j. ejmech.2022.114859; b) M. Carcelli, M. Tegoni, J. Bartoli, C. Marzano, G. Pelosi, M. Salvalaio, D. Rogolino, V. Gandin, Eur. J. Med. Chem. 194 (2020), 112266, https://doi.org/10.1016/j.ejmech.2020.112266.
[19] a) M. A. Alhasania, T. A. Farghaly, H. A. El-Ghamry, J. Mol. Struct. 1249 (2022), 131607, https://doi.org/10.1016/j.molstruc.2021.131607; b) H. A. El-Ghamry, K. Yamauchi, K. Sakai, T. A. Farghaly, Inorg. Chim. Acta. 516 (2021), 120117, https://doi.org/10.1016/j.ica.2020.120117; c) K. M. Takroni, T. A. Farghaly, M. F. Harras, H. A. El-Ghamry, Applied organometallic Chemistry 17. https://doi.org/10.1002/aoc.5860, June 2020.
[20] a) E. Urso, M. Maffia, J. Vase. Res 52 (2015) 172-196; b); c) S. A. Lowndes, A. L. Harris, J. Mammary Gland Biol. Neoplasia 10 (2005) 299-310.
[21] a) Å. Kellett, Z. Molphy, V. McKee, C. Slator, Recent advances in anticancer copper complexes, in: I. A. Vessieres, S. M. Meier-Menches, A. Casini (Eds.), Metal-Based Anticancer Agents, 14, RSC Metallobiology, London, U K, 2019, pp. 91-119; b) Å. Hordyjewska, L. Popiolek, J. Kocot, Biometals 27 (2014) 611-621.
[22] a) M. Wehbe, M. Anantha, M. Shi, A. Leung, W. H. Dragowska, L. Sanche, M. B. Bally, Int. J. Nanomed. 12 (2017) 4129-4146, https://doi.org/10.2147/IJN. S137347; b) X. Li, K. Du, J. Sun, F. Feng, ACS Appl. Bio Mater. 3 (2020) 654-663, https://doi.org/10.1021/acsabm.9b01014; c) B. Cvek, V. Milacic, J. Taraba, Q. P. Dou, J. Med. Chem. 51 (2008) 6256-6258, https://doi.org/10.1021/jm8007807. d)H. Liu, Y. Kong, Z. Liu, X. Guo, B. Yang, T. Yin, H. He, J. Gou, Y. Zhang, X. Tang, Int. J. Pharm. 621 (2022), 121788. doi: 10.1016/j.ijpharm.2022.121788.
[23] a) S. Tardito, A. Barilli, I. Bassanetti, M. Tegoni, O. Bussolati, R. Franchi-Gazzola, C. Mucchino, L. Marchio, J. Med. Chem. 55 (2012) 10448-10459, https://doi.org/10.1021/jm301053a; b) S. M. Zhai, L. Yang, Q. C. Cui, Y. Sun, Q. P. Dou, B. Yan, J. Biol. Inorg. Chem. 15 (2010) 259-269, https://doi.org/10.1007/s00775-009-0594-5.
[24]N. N. Liu, C. J. Liu, X. F. Li, S. Y. Liao, W. B. Song, C. S. Yang, C. Zhao, H. B. Huang, L. X. Guan, P. Q. Zhang, S. T. Liu, X. L. Hua, X. Chen, P. Zhou, X. Y. Lan, S. G. Yi, S. Q. Wang, X. J. Wang, Q. P. Dou, J. B. Liu. Sci Rep 4 (2014) 5240, https://doi.org/10.1038/Srep05240.
[25] a) S. Nazeem, A. S. Azmi, S. Hanif, A. Ahmad, R. M. Mohammad, S. M. Hadi, K. S. Kumar, Mutagenesis 24 (2009) 413-418, https://doi.org/10.1093/mutage/gep023; b) Z. F. Chen, M. X. Tan, L. M. Liu, Y. C. Liu, H. S. Wang, B. Yang, Y. Peng, H. G. Liu, H. Liang, C. Orvig, Dalton Trans. 48 (2009) 10824-10833.
[26] a) M. Wehbe, A. K. Malhotra, M. Anantha, C. Lo, W. H. Dragowska, N. D. Santos, M. B. Bally, Drug Deliv Transl Res 8 (2018) 239-251, https://doi.org/10.1007/s13346-O17-0455-7; b) Å.D. Schimmer, Curr. Cancer Drug Targets 11 (2011) 325-331; c) W. Q. Ding, B. L. Liu, J. L. Vaught, H. Yamauchi, S. E. Lind, Cancer Res. 65 (2005) 3389-3395; d) K. G. Daniel, D. Chen, S. Orlu, Q. C. Cui, F. R. Miller, Q. P. Dou, Breast cancer research, BCR 7 (2005) R₈₉₇-R₉₀₈, https://doi.org/10.1186/bcr1322.
[27] a) D. Hedley, A. Shamas-Din, S. Chow, D. Sanfelice, A. C. Schuh, J. M. Brandwein, M. D. Seftel, V. Gupta, K. W. L. Yee, A. D. Schimmer, Leuk. Lymphoma 57 (2016) 2437-2440A; b) L. Ruiz-Azuara, G. Bastian, M. E. Bravo-G as, M. Flores-Alamo, I. Fuentes, C. Mejia, J. C. Garcia-Ramos, A. Serrano, Cancer Res. 74 (2014). CT408—CT408. Abstract CT408.
[28] a) L. Schwartz, C. T. Supuran, K. O. Alfarouk, Med. Chem. 17 (2017) 164-170; b) C. Marzano, M. Pellei, F. Tisato, C. Santini, Med. Chem. 9 (2009) 185-211; b) T. F. Kagawa, B. H. Geierstanger, A. H. J. Wang, P. S. Ho, J. Biol. Chem. 266 (1991), 20175-2018.
[29] a) D. Palanimuthu, S. V. Shinde, K. Somasundaram, A. G. Samuelson, J. Med. Chem. 56 (2013) 722-734; b) N. Raman, R. Jeyamurugan, B. Rajkapoor, V. Magesh, Appl. Organomet. Chem. 23 (2009) 283-290.
[30] a) N. Graf, S. J. Lippard, Adv. Drug Deliv. Rev. 64 (2012) 993-1004; A. M. da Costa Ferreira, J. Inorg. Biochem. 186 (2018) 85-94.
[31]W. J. Geary, Coord. Chem. Rev. 7 (1971) 81-122.
[32] ek, R. C. Fischerc, F. A. Mautner, RSC Adv. 5 (2015) 87139-87150.
[33]N. Hendrickson, J. M. Latour, Inorg. Chem. 30 (1991) 1357-1365.
[34]B. J. Hathaway, G. Wilkinson, R. D. Gillard, in: J. A. McCleverty (Ed.), In Comprehensive Coordination Chemistry, 5, Pergamon, Press, Oxford, England, 1987, p. 533.
[35] Polyhedron 124 (2017) 237-242; C. Fischer, F. A. Mautner, Polyhedron 90 (2015) 258-265.
[36] a) S. S. Massoud, A. E. Guilbeau, H. T. Luong, R. Vicente, J. H. Albering, R. C. Fischer, F. A. Mautner, Polyhedron 54 (2013) 26-33; b) F. A. Mautner, J. B. Soileau, P. K. Bankole, A. A. Gallo, S. S. Massoud, J. Mol. Struct. 889 (2008) 271-278.
[37]A. W. Addison, T. N. Rao, J. Reedijk, J. V. Rijin, G. C. Verschoor, J. Chem, Soc, Dalton Trans. (1984) 1349-1356.
[38]K. Gaur, A. M. V′azquez-Salgado, G. Duran-Camacho, I. Dominguez-Martinez, J. A. Benjamin-Rivera, L. Fern′andez-Vega, L. Carmona Sarabia, A. Cruz Garcia, F. P′erez-Deliz, J. A. M‘endez Rom’an, M. Vega-Cartagena, S. A. Loza-Rosas, X. Rodriguez Acevedo, A. D. Tinoco, INORGA 6 (2018) 126, https://doi.org/10.3390/inorganics6040126.
[39]A. Steinbrueck, A. C. Sedgwick, J. T. Brewster II, K.—Ch Yan, Y. Shang, D. M. Knoll, G. iga, X.-P. He, H. Tian, J. L. Sessler, Chem. Soc. Rev. 49 (2020) 3726-3747.
[40]C. Molinaro, A. Martoriati, L. Pelinski, K. Cailliau, Cancers 12 (2020) 2863.
[41] ales-Ballesteros, C. Mejia, L. Ruiz-Azuara, FEBS Open Bio 12 (2022) 880-899.
[42]C. Molinaro, N. Wambang, T. Bousquet, A. S. Vercoutter-Edouart, L. Polinski, K. Cailliau, A. Martoriati, Front. Oncol. 12 (2022) 837373.
[43]R. A. Khan, M. R. Khan, M. Usman, F. Sayeed, H. A. Alghamdi, S. Alrumman, W. Alharbi, N. N. Farshori, M. M. Al-Oqail, M. R. Siddiqui, M. A. Khanjer, A. Alsalme, Saudi J. Biol. Sci. 27 (2020) 2164-2173.
[44]F. Janku, D. J. McConkey, D. S. Hong, R. Kurzrock, Nat. Rev. Clin. Oncol. 8 (2011) 528-539.
[45]C. Molinaro, N. Wambang, T. Bousquet, A. S. Vercoutter-Edouart, L. Polinski, K. Cailliau, A. Mortoriati, Front. Oncol. 12 (2022), 837373.
[46]M. Kocak, S. M. Corsello, S. Lutsenko, N. Kanarek, S. Santagata, T. R. Golub, Science 375 (2022) 1254-1261.
[47] a) Bruker, SAINT V. 7.23; Bruker (2006) APEX 2 2.0-2, Bruker AXS Inc., Madison, ; b) G. M. Sheldrick, SADABS v. 2, University of Goettingen, Germany, 2001.
[48] a) G. M. Sheldrick, Acta Crystallogr. A 64 (2008) 112; b) G. M. Sheldrick, Acta Crystallogr, C 71 (2015) 3.
[49] a) C. F. Macrae, P. R. Edington, P. McCabe, E. Pidcock, G. P. Shields, R. Taylor, T. Towler, J. van de Streek, J. Appl. Crystallogr. 39 (2006) 453; b) Å.L. Speck, PLATON, a Multipurpose Crystallographic Tool, Utrecht University, The Netherlands, 2000.

What is claimed is:

1. A phenoxido copper compound or a complex, hydrate, solvate, geometric isomer, or salt thereof, wherein the compound has a chemical structure selected from a list: (Cu₂(L¹)₂](ClO₄)₂;
(Cu₂(L²)₂](ClO₄)₂; [Cu₂(L³)₂]; [Cu₂(L₄)₂]; [Cu₂(L⁴)₂]; [Cu₂(L⁵)₂(H₂O)]·2H₂O;
[Cu₂(L⁶)₂(H₂O)]·2H₂O; catena-[Cu(—L⁷)], wherein L¹⁻⁷ is a ligand with a chemical structure:

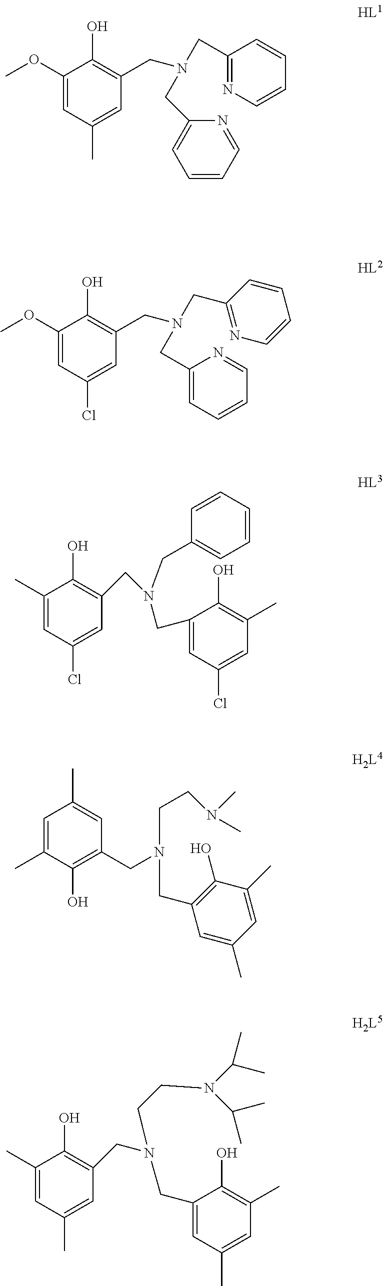

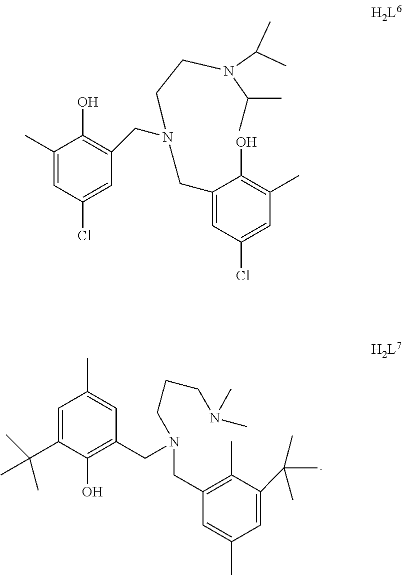

2. The phenoxido copper compound or a complex, hydrate, solvate, geometric isomer, or salt thereof of claim 1, wherein L is a ligand with a chemical structure:

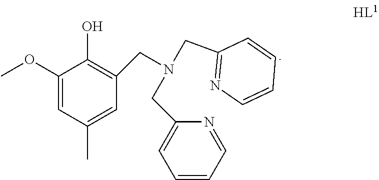 [SMILES: COc1cc(C)cc(CN(Cc2ccccn2)Cc2ccccn2)c1O]

3. The phenoxido copper compound or a complex, hydrate, solvate, geometric isomer, or salt thereof of claim 1, wherein L is a ligand with a chemical structure:

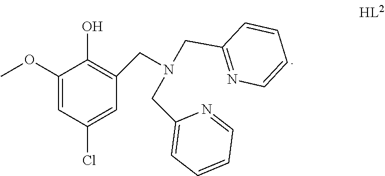 [SMILES: COc1cc(Cl)cc(CN(Cc2ccccn2)Cc2ccccn2)c1O]

4. The phenoxido copper compound or a complex, hydrate, solvate, geometric isomer, or salt thereof of claim 1, wherein L is a ligand with a chemical structure:

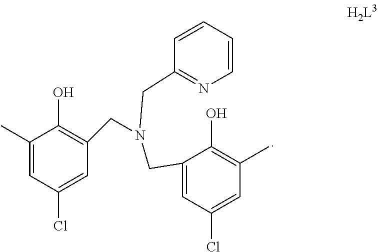

5. The phenoxido copper compound or a complex, hydrate, solvate, geometric isomer, or salt thereof of claim 1, wherein L is a ligand with a chemical structure:

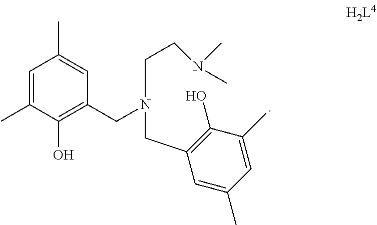

6. The phenoxido copper compound or a complex, hydrate, solvate, geometric isomer, or salt thereof of claim 1, wherein L is a ligand with a chemical structure:

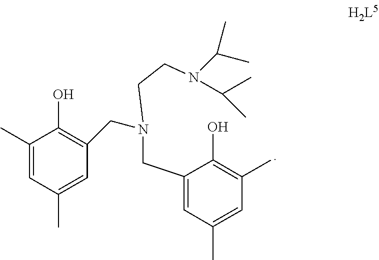

7. The phenoxido copper compound or a complex, hydrate, solvate, geometric isomer, or salt thereof of claim 1, wherein L is a ligand with a chemical structure:

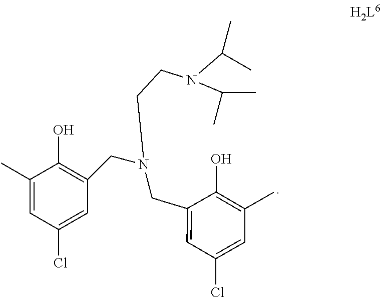

8. The phenoxido copper compound or a complex, hydrate, solvate, geometric isomer, or salt thereof of claim 1, wherein L is a ligand with a chemical structure:

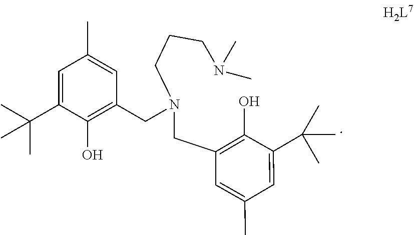 [SMILES: Cc1cc(CN(CCCN(C)C)Cc2cc(C)cc(C(C)(C)C)c2O)c(O)c(C(C)(C)C)c1]

9. A method for treating cancer, wherein the method comprises administering to a subject a therapeutically effective amount of a phenoxido copper compound or a complex, hydrate, solvate, geometric isomer, or salt thereof with a chemical structure selected from a list: (Cu₂(L¹)₂](ClO₄)₂; (Cu₂(L²)₂](ClO₄)₂; [Cu₂(L³)₂]; [Cu₂(L⁴)₂]; [Cu₂(L⁴)₂];
[Cu₂(L⁵)₂(H₂O)]·2H₂O; [Cu₂(L⁶)₂(H₂O)]·2H₂O; catena-[Cu(—L⁷)], wherein L-⁷ is a ligand with a chemical structure:

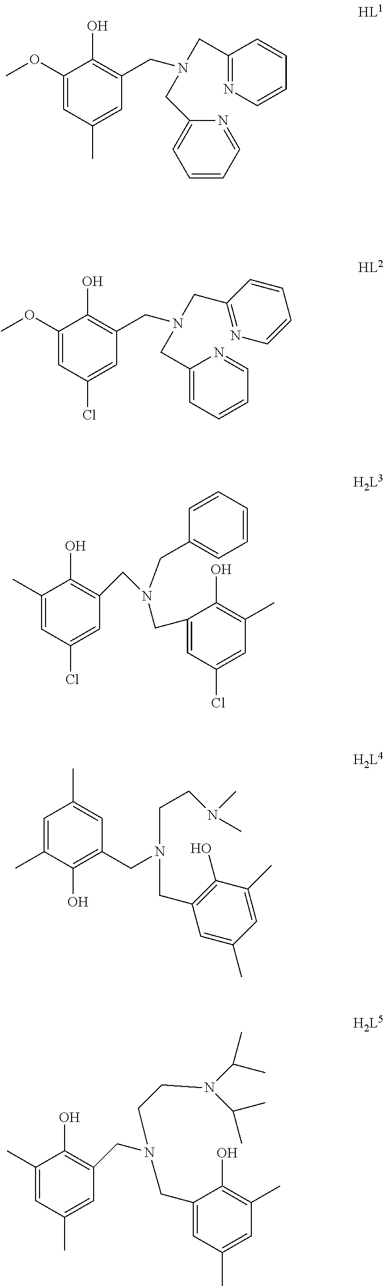

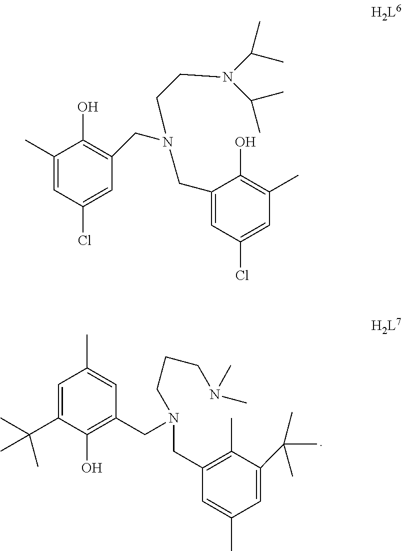

10. The method for treating a cancer of claim 9, wherein Lis a ligand with a chemical structure:

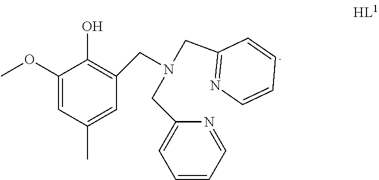 [SMILES: COc1cc(C)cc(CN(Cc2ccccn2)Cc2ccccn2)c1O]

11. The method for treating a cancer of claim 9, wherein Lis a ligand with a chemical structure:

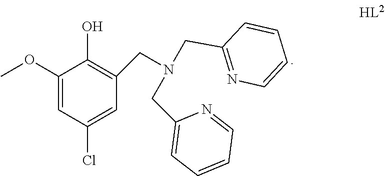 [SMILES: COc1cc(Cl)cc(CN(Cc2ccccn2)Cc2ccccn2)c1O]

12. The method for treating a cancer of claim 9, wherein L is a ligand with a chemical structure:

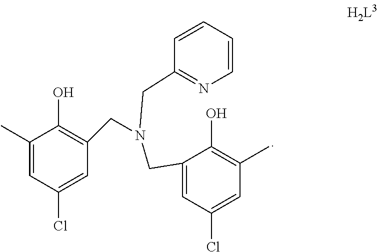

13. The method for treating a cancer of claim 9, wherein Lis a ligand with a chemical structure:

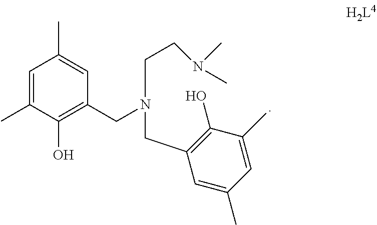

14. The method for treating a cancer of claim 9, wherein Lis a ligand with a chemical structure:

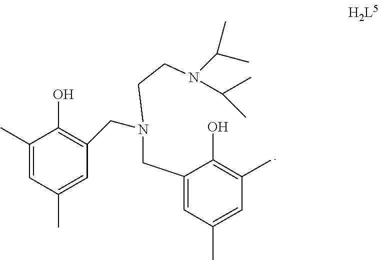

15. The method for treating a cancer of claim 9, wherein Lis a ligand with a chemical structure:

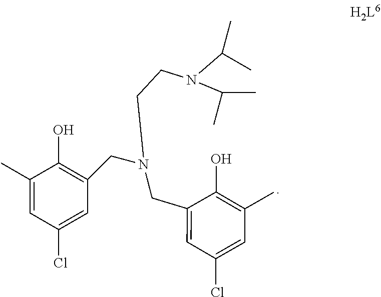

16. The method for treating a cancer of claim 9, wherein Lis a ligand with a chemical structure:

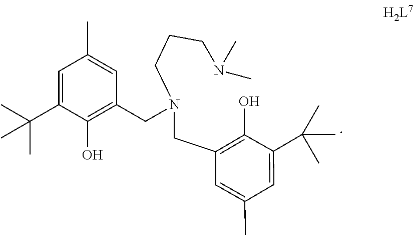 [SMILES: Cc1cc(CN(CCCN(C)C)Cc2cc(C)cc(C(C)(C)C)c2O)c(O)c(C(C)(C)C)c1]

17. The method for treating a cancer of claim 9, wherein the cancer is an ovarian cancer.

18. The method for treating a cancer of claim 10, wherein the cancer is an ovarian cancer.

19. The method for treating a cancer of claim 11, wherein the cancer is an ovarian cancer.

20. The method for treating a cancer of claim 12, wherein the cancer is an ovarian cancer.
